(19) United States
(12) Patent Application Publication
(10) Pub. No.: US 2025/0255820 A1
(43) Pub. Date: Aug. 14, 2025

(54) COMPOSITIONS COMPRISING INDIGO AND/OR AN INDIGO DERIVATIVE AND METHODS OF USE THEREOF

(71) Applicant: Azora Therapeutics, Inc., Encino, CA (US)

(72) Inventors: Julie Patricia Saiki, Redwood City, CA (US); Matthew Davidson, Encino, CA (US); Johan Oscar Lennart Andreasson, Redwood City, CA (US); Michael David Favero, Tampa, FL (US); Matthew Benjamin Greene, Tampa, FL (US)

(21) Appl. No.: 19/053,798

(22) Filed: Feb. 14, 2025

Related U.S. Application Data
(63) Continuation of application No. 17/727,510, filed on Apr. 22, 2022, now abandoned.
Application No. 17/727,510 is a continuation of application No. 16/865,183, filed on May 1, 2020, now abandoned.
(60) Provisional application No. 62/843,184, filed on May 3, 2019.

(51) Int. Cl. A61K 9/20; A61K 9/28; A61K 31/404

(52) U.S. Cl. CPC A61K 9/2054; A61K 9/284; A61K 31/404

(57) ABSTRACT

Compositions comprising an AhR agonist compound, such as indigo and/or an indigo derivative, such as indirubin and isatin, are described. Methods of treatment, including the treatment of ulcerative colitis, by administering the compositions are described. In an embodiment, compositions in the form of a solid amorphous dispersion of the AhR agonist are described.

### CROSS-REFERENCE TO RELATED APPLICATIONS

This application is a continuation application of U.S. application Ser. No. 17/727,510, filed Apr. 22, 2022, pending, which is a continuation of U.S. application Ser. No. 16/865,183, filed May 1, 2020, now abandoned, which claims the benefit of U.S. Provisional Application No. 62/843,184, filed May 3, 2019, now expired, each of which is incorporated by reference herein in its entirety.

### TECHNICAL FIELD

The subject matter described herein relates to compositions comprising indigo and/or an indigo derivative, such as indirubin, and to methods of treatment, including the treatment of inflammatory bowel diseases.

### BACKGROUND

Indigo naturalis (Qing Dai) is a plant extract prepared from the plants Indigofera tinctoria, Indigofera suffruticosa, Polygonum tinctorium, and/or Isatis indigotica. An exemplary Indigo naturalis composition is an extract of Baphicacanthus cusia (Nees) Bremek of the Acanthacaea family. Indigo naturalis is composed of multiple compounds known to activate the aryl hydrocarbon receptor. Bespoke compositions offering the benefits of Indigo naturalis with optimized clinical efficacy and safety are desired. In particular, oral dosage forms with one or more of the active components of Indigo naturalis, such as the indigo derivative indirubin, are desired.

It is known that indigo and indirubin are potent agonists of the aryl hydrocarbon receptor. Indigo is less potent than indirubin at activating the aryl hydrocarbon receptor, requiring more drug to achieve the same effect. Both compounds have poor aqueous dissolution and solubility, making it difficult to formulate suitable pharmaceutical dosage forms. Also, the limited dissolution and solubility of indirubin presents drug delivery challenges especially when targeting local drug delivery to the gastro-intestinal tract. Mechanisms to make indirubin locally but not systemically bioavailable are desired for the treatment of inflammatory bowel diseases. Methods to activate the AhR in the colon or intestine, but not systemically in organs such as the liver or lungs, may minimize potential side effects like changes in liver enzymes and the development of pulmonary arterial hypertension.

### BRIEF SUMMARY

The following aspects and embodiments thereof described and illustrated below are meant to be exemplary and illustrative, not limiting in scope.

In a first aspect, a composition comprising a compound selected from indigo, an indigo derivative, and combinations thereof and a pharmaceutically acceptable carrier is provided. The composition provides a therapeutic effect with respect to treating ulcerative colitis that is substantially the same as provided by a composition of Indigo naturalis. In another embodiment, the composition provides a synergistic or a superior therapeutic effect with respect to treating ulcerative colitis as compared to the effect provided by a composition of Indigo naturalis. In still another embodiment, the composition provides a reduction in observed side effects compared to the side effects observed following administration of Indigo naturalis. In yet another embodiment, the composition provides a synergistic effect with respect to treating ulcerative colitis as compared to the effect provided by a composition of the carrier and indigo or an indigo derivative individually.

In one embodiment, the composition comprises a compound selected from indigo, indirubin, isatin, and combinations thereof. In one embodiment, the composition comprises only or solely active therapeutic compound selected from indigo and indigo derivatives, or selected from indigo, indirubin, isatin, and combinations thereof.

In one embodiment, the composition is not Indigo naturalis, the known plant extract.

In one embodiment, the compound in the composition is synthetically produced. In another embodiment, the compound is obtained from a plant extract or wherein the compound is within or part of a plant extract.

In another embodiment, the composition comprises solely or only indigo, indirubin or isatin. In another embodiment, the composition comprises solely or only an indigo derivative.

In another aspect, a dosage form is provided that is comprised of a solid dispersion of an aryl hydrocarbon receptor agonist and a polymeric hydrophilic carrier, wherein aryl hydrocarbon receptor agonist is in a substantially amorphous form in the dispersion.

In yet another aspect, a formulation is provided that is comprised of a solid dispersion of an aryl hydrocarbon receptor agonist and a polymeric hydrophilic carrier, wherein aryl hydrocarbon receptor agonist is in a substantially amorphous form in the dispersion; and one or more excipients in an amount greater than about 20 wt % of the formulation and blended with the solid dispersion to form a blend. In one embodiment, the one or more excipients is/are present in the formulation in an amount greater than about 25 wt %, 30 wt %, 35 wt %, 40 wt %, 45 wt %, 50 wt %, 55 wt %, 60 wt %, 65 wt % or 70 wt %.

In an embodiment, the aryl hydrocarbon receptor is natural indirubin or synthetic indirubin.

In another embodiment, the dispersion comprises between about 0.1-30 wt %, 0.5-30 wt %, 0.5-25 wt %, 0.5-20 wt %, 0.5-15 wt %, 1-25 wt %, 1-20 wt %, 2-25 wt %, 2-20 wt % or 2015 wt % or 2-10 wt % of the aryl hydrocarbon receptor, such as natural or synthetic indirubin.

In an embodiment, the dispersion comprises a ratio of polymeric hydrophilic carrier to indirubin of 10:1 to 50:1. In another embodiment, the dispersion comprises a ratio of polymeric hydrophilic carrier to indirubin of 10:1 to 25:1.

In some embodiments, the polymeric hydrophilic carrier is a cellulosic polymer, a cellulosic copolymer, a polyvinyl acetate polymer, a polyvinyl acetate-polyethylene glycol copolymer, a methacrylic acid copolymer, a methacrylic acid-methyl methacrylate copolymer, a polyvinyl pyrrolidone polymer, a polyvinyl pyrrolidone copolymer, a polyvinyl alcohol polymer, or a polyvinyl alcohol copolymer.

In another embodiment, the cellulosic polymer is a synthetic cellulosic polymer selected from hydroxypropyl methylcellulose acetate succinate, hydroxypropyl methylcellulose phthalate, hydroxypropyl methylcellulose and carboxymethyl ethylcellulose.

In one embodiment, the methacrylic acid-methyl methacrylate copolymer is a 1:2 or a 1:1 copolymer of methacrylic acid and methyl methacrylate.

In another embodiment, the cellulosic polymer is selected from cellulose acetate phthalate and hydroxypropyl methylcellulose phthalate.

In yet another embodiment, the polymeric hydrophilic carrier dissolves or becomes soluble at a pH between 4.5-7.5 in the intestinal tract or in simulated gastric fluid at a temperature of 25° C. or 30° C. or 35° C. In another embodiment, the polymeric hydrophilic carrier dissolves or becomes soluble at a pH between 4.5-7.5 and is insoluble at pH 1.5-3.5 in the intestinal tract or in simulated gastric fluid at a temperature of 25° C. or 30° C. or 35° C.

In other embodiments, the dosage form or the formulation achieves one or more of (i) the release of substantially all of the indirubin from the dosage form in the small intestine, large intestine and/or colon; and (ii) the release of substantially all indirubin from the dosage form within about 60 minutes when at a pH above 5; (iii) the release of substantially all of the indirubin from the dosage form over a period of about 8 hours when at a pH of above 5

In other embodiments, one or more of (i), (ii) or (iii) is achieved without use of an enteric polymer coating on the outside of the dosage form.

In another embodiment, one or more of (i), (ii) or (iii) is achieved with the use of an enteric polymer coating on the outside of the dosage form.

In other embodiments, the dispersion has an indirubin solubility in simulated intestinal fluid greater than a dispersion containing the same amount of indirubin in the form of crystalline indirubin, Indigo naturalis or a 1:9 povidone:indirubin solid dispersion in simulated intestinal fluid, at the same temperature.

In other embodiments of the dosage form or the formulation, the indirubin remains in a substantially amorphous form the dispersion for at least about 6 months when stored at 25° C.

In other embodiments of the dosage form or the formulation, the dispersion does not contain indigo, and/or wherein the dosage form does not contain indigo.

In other embodiments of the dosage form or the formulation, the hydrophilic carrier is not hypromellose acetate succinate and/or wherein the solid dispersion is not produced via precipitation from dimethylacetamide. In other embodiments of the dosage form or the formulation, the polymeric hydrophilic carrier is not povidone.

In other embodiments of the dosage form or the formulation, when administered it provides a therapeutic effect with respect to treating ulcerative colitis that is equal or superior to the therapeutic effect provided by an oral composition of Indigo naturalis containing an equivalent amount of indirubin. For example, in an embodiment, the dosage form or formulation provides a reduction in observed side effects compared to the side effects observed following administration of Indigo naturalis.

In one embodiment, the dosage form or formulation is suitable for localized delivery. In an embodiment, the localized delivery via oral administration to the small intestine or colon. In another embodiment, the localized delivery is topical delivery to intact or disrupted oral mucosa. In another embodiment, the localized delivery is topical delivery to the rectum or colon.

In one embodiment, the dosage form or formulation yields a non-therapeutic blood level of indirubin.

In one embodiment, the dosage form or formulation yields a systemic blood level of less than about 1 nM of the compound.

In one embodiment, the dosage form, or the formulation, is a tablet, a capsule, a gel cap, an enteric-coated tablet, an enteric-coated tablet capsule, an enteric-coated gel cap, a collection of microspheres, a collection of nanoparticles, a suspension, a powder for suspension, an orally-disintegrating tablet, a buccal tablet, an orally-dissolving film, a lozenge, a suppository, foam, an enema, an ointment, a cream, or a gel.

In an embodiment, the one or more excipients is selected from the group consisting of a disintegrant, a filler, a surfactant, and a lubricant.

In still other embodiments, the formulation or dosage form comprises a carrier that is suitable for oral administration.

In an embodiment, the carrier is a polymer that provides for release of a majority of the compound or of at least two compounds in the colon or in the stomach or in the small intestine. In exemplary embodiments, the polymer is a pH-sensitive polymer. In another embodiment, the polymer solubility increases with increasing pH. Exemplary polymers, in some embodiments, are polyvinyl polymers, cellulosic polymers, polyvinyl-polyethylene glycol copolymers, methacrylic acid copolymers, and methacrylic acid-methyl methacrylate copolymers. The copolymer can be a 1:2 or a 1:1 copolymer of methacrylic acid and methyl methacrylate. The cellulosic polymer can be selected from cellulose acetate phthalate and hydroxypropyl methylcellulose phthalate.

In other embodiments, the polymer is a biodegradable polymer. An example is a polymer that degrades in the presence of hydrolytic or metabolizing enzymes, such as a polysaccharide or an azo-aromatic polymer. In embodiments, the polysaccharide is selected from chitosan, guar gum, xanthan gum, dextran, pectin, amylose, inulin, chondroitin sulfates, and derivatives thereof. In other embodiments, the azo-aromatic polymer is prepared from a monomer selected from the group consisting of acrylic acid, butyl methacrylate, methyl methacrylate, styrene, and hydroxyethyl methacrylate. In still other embodiments, the azo-aromatic polymer is a copolymer of two or more of the monomers.

In other embodiments, the polymer is a bioadhesive/gastroretentive polymer or a hydrogel. In embodiments, the bioadhesive/gastroretentive polymer is selected from the group consisting of polycarbophils, polyurethanes, and polyethylene oxides.

In another embodiment, the compound or at least two compounds are formulated alone or with a carrier into particles or granules. The particles or granules, in one embodiment, comprise a population of particles or granules with a size distribution tailored to achieve delayed or extended release of the compound(s).

In other embodiments, the polymers allow for immediate or slow release of the active agents. In some embodiments the polymer is selected from a group consisting of alginates, cyclodextrins, polyglycolides or polylactides.

In other embodiments, the formulation contains a penetration enhancer to allow for penetration into the intestinal tissue or through the intestinal tissue into the blood stream.

In other embodiments, wherein the carrier is suitable for rectal administration.

In other embodiments, the carrier is suitable for topical administration. Exemplary topical compositions include, but are not limited to, a gel, a suspension, a lozenge, a cream, a foam, a suppository, an enema or an ointment.

In another aspect, a method for treating ulcerative colitis (UC) comprises administering to a subject a therapeutically effective amount of a composition comprising indigo, indirubin, isatin, or a combination thereof, wherein the composition is not Indigo naturalis, whereby the administering treats UC in the subject.

In yet another aspect, a method for treating inflammatory bowel disease in a subject comprises administering to a subject a therapeutically effective amount of a composition comprising indigo, indirubin, isatin, or a combination thereof, wherein the composition is not Indigo naturalis, whereby the administering treats the inflammatory bowel disease in the subject. In an embodiment, the inflammatory bowel disease is not Crohn's disease.

In still another aspect, a method for preventing recurrence of UC comprises administering to a subject a therapeutically effective amount of a composition comprising indigo, indirubin, isatin, or a combination thereof, wherein the composition is not Indigo naturalis, whereby the administering prevents recurrence of UC in the subject.

In still another aspect, a method for treating UC in a subject previously treated with 5-aminosalicylic acid or a prodrug of 5-aminosalicylic acid comprises administering to a subject a therapeutically effective amount of a composition comprising indigo, indirubin, isatin, or a combination thereof, wherein the composition is not Indigo naturalis, whereby the administering treats UC in the subject.

In another aspect, a method for treating ulcerative colitis (UC) in a subject refractory to treatment with 5-aminosalicylic acid, a prodrug of 5-aminosalicylic acid, a steroid, small molecule immunosuppressants (e . . . g, azathioprine, 6-mercaptopurine, methotrexate), an anti-TNF-alpha antibody (e.g., infliximab, adalimumab), a Janus kinase inhibitor (e.g., tofacitinib, peficitini), an integrin receptor antagonist (e.g., vedolizumab, etrolizumab, an alpha-4 beta-7 integrin antagonist), an anti-IL-12/23 antibody (ustekinumab), a sphingosine-1-phosphate receptor modulator (e.g., ozanimod), or any other known drug therapy for UC is provided. The method comprises administering to a subject a therapeutically effective amount of a composition comprising indigo, indirubin, isatin, or a combination thereof, wherein the composition is not Indigo naturalis, whereby the administering treats UC in the subject.

In another aspect, a method for treating mild to moderate UC in a subject refractory to treatment with 5-aminosalicylic acid or a prodrug of 5-aminosalicylic acid, the patient untreated with an anti-TNF-alpha antibody, comprises administering to a subject a therapeutically effective amount of a composition comprising indigo, indirubin, isatin, or a combination thereof, wherein the composition is not Indigo naturalis, whereby the administering treats UC in the subject.

In embodiments of the foregoing methods, the subject has moderate to severe UC. In other embodiments, the subject has mild to moderate UC. In other embodiments, the subject has mild to moderate Crohn's disease or moderate to severe Crohn's disease. In other embodiments the subject has gastrointestinal graft-vs-host disease, pouchitis, or mucositis.

In embodiments, of the foregoing methods treatment re-sensitizes the subject to be responsive to previously failed or ineffective therapies.

In addition to the exemplary aspects and embodiments described above, further aspects and embodiments will become apparent by study of the following description.

Additional embodiments of the present methods and compositions, and the like, will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples.

### BRIEF DESCRIPTION OF THE DRAWINGS

FIGS. 1A-1B are images of solid dispersions of synthetic indirubin and a hydrophilic polymer, polyvinylpyrrolidone, where the indirubin is not amorphous.

FIGS. 2A-2B are images of amorphous solid dispersions of of synthetic indirubin and a hydrophilic polymer, polyvinylpyrrolidone.

FIG. 3A is an x-ray diffractogram of the empty zero background sample holder, as a reference.

FIG. 3B is an x-ray diffractogram for 100% crystalline Indirubin (at a concentration equivalent to 10% drug load samples), as a comparative reference.

FIG. 3C is an x-ray diffractogram of a dispersion of 10% indirubin in PVP (povidone).

FIG. 3D is an x-ray diffractogram of a dispersion of 5% indirubin in PVP.

FIG. 3E is an x-ray diffractogram of a dispersion of 10% indirubin in polyvinylpyrrolidone-vinyl acetate copolymer (copovidone).

FIG. 3F is an x-ray diffractogram of a dispersion of 5% indirubin in polyvinylpyrrolidone-vinyl acetate copolymer (copovidone).

FIG. 3G is an x-ray diffractogram of a dispersion of 10% indirubin in hydroxypropyl methylcellulose acetate succinate (HPMC-AS).

FIG. 3H is an x-ray diffractogram of a dispersion 5% indirubin in hydroxypropyl methylcellulose acetate succinate (HPMC-AS).

FIG. 3I is an x-ray diffractogram of a dispersion of 10% indirubin in hydroxypropylmethylcellulose phthalate (HPMCP).

FIG. 3J is an x-ray diffractogram of a dispersion of 5% indirubin in hydroxypropylmethylcellulose phthalate (HPMCP).

FIG. 3K is an x-ray diffractogram of a dispersion of 10% indirubin in polyvinyl acetate phthalate (PVAP).

FIG. 3L is an x-ray diffractogram of a dispersion of 5% indirubin in polyvinyl acetate phthalate (PVAP).

FIG. 3M is an x-ray diffractogram of a dispersion of 10% indirubin in PVAc-PVCap-PEG, (SOLUPLUS®).

FIG. 4 is a graph showing the fold improvement in activation of the human AhR in vitro for indirubin, indigo and Indigo naturalis as a function of concentration, in ng/mL.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the topical compositions and all measurements made are at about 25° C., unless otherwise specified. Thus “wt %” refers to the value determined by dividing the weight of the portion of a formulation or composition by the total weight of that formulation or composition, multiplied by 100.

All percentages, parts and ratios are based upon the total weight of the topical compositions and all measurements made are at about 25° C., unless otherwise specified.

A “dispersion” refers to an admixture or system of particles of one material essentially homogeneously dispersed in a continuous phase of a second material. In one embodiment, the particles are drug (active agent) particles and the second material is a polymer, such as a copolymer of methacrylic acid.

The term “indigo,” as used herein, generally refers to a compound having the formula below identified as “indigo.”

An “indigo derivative” refers to derivatives of indigo such as those exemplified by the compounds identified below as leuco-indigo, indirubin, meisoindigo, and isatin (an oxidized version of half of the indigo molecule):

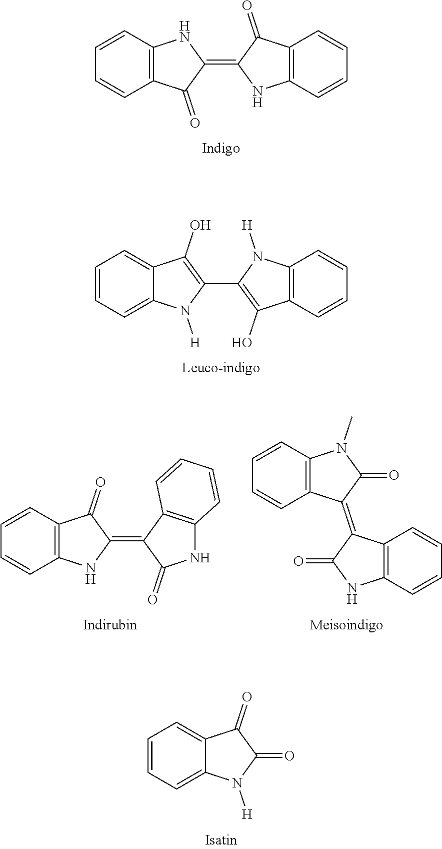

Additional non-limiting examples of indigo derivatives include thioindigo, indigo carmine, indirubin-3′-monoxime, indirubin-5-sulphonic acid or 5-chloro-indirubin, bromoindirubin-3-oxime, 5-halogenoindirubin, N-ethyl-indirubin, N-methylisoindigo, the compounds set forth in U.S. Patent Application Publication No. 2017/0304381, which is incorporated by reference herein, the compounds set forth in Hubbard, T. D. et al., Drug Metabolism and Disposition, 43:1522-1535 (2015), which is incorporated by reference herein. The term “indigo derivative” excludes prodrugs of indigo and indigo derivatives unless express reference is made to “prodrug of indigo” or “prodrug of an indigo derivative.”

“Indigo naturalis” refers to a plant extract that contains indigo and other indigo derivatives. This extract can be crude or highly purified to enrich for indigo and/or an indigo derivative. The plant extract can be from the species Indigofera tinctoria, Indigofera suffruticosa, Polygonum tinctorium, Isatis indigotica, Baphicacanthus cusia or other plants or yeast or bacteria that contain indigo.

The terms “inhibiting” or “reducing” are used in reference to methods to inhibit or to reduce a clinical symptom of a disorder in a population with the disorder as compared to an untreated, control population of subjects with the disorder.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

Pharmaceutically acceptable salt” denotes a salt form of a drug or active ingredient, or other ingredient having at least one group suitable for salt formation that causes no significant adverse toxicological effects to the patient. Reference to an AhR agonist compound, such as indirubin, is meant to encompass its pharmaceutically acceptable salts, as well as solvates and hydrates thereof. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitable pharmaceutically acceptable salt forms and methods for identifying such salts are found in, e.g., Handbook of Pharmaceutical Salts: Properties, Selection and Use, Weinheim/Zürich: Wiley—VCH/VHCA, 2002; P. H. Stahl and C. G. Wermuth, Eds. As used herein, the phrases “pharmaceutically acceptable excipient” and

“pharmaceutically acceptable carrier” refer to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like.

As used herein, a “pH sensitive coating” is a layer applied to or enclosing a capsule or tablet that is stable in one pH range, yet becomes unstable, or permeable, or soluble, in another pH range. For example, many pH sensitive coatings are insoluble at low pH (e.g., near pH 3) yet become more soluble at higher pH (e.g., above pH 5 or 6). Such a coating may also be termed an “enteric coating”.

As used herein, the terms “patient” or “subject” refers to a living organism suffering from or prone to a condition that can be treated by administration of a pharmaceutical compositions as provided herein. Non-limiting examples include humans, other mammals and other non-mammalian animals.

“Substantially” or “essentially” means nearly totally or completely, for instance, 95% or greater, of a given quantity.

The term “treating” is used herein, for instance, in reference to methods of treating an inflammatory disorder, and generally includes the administration of a compound or composition which reduces the frequency of, or delays the onset of, symptoms of the medical condition (e.g., UC) in a subject relative to a subject not receiving the compound or composition. This can include reversing, reducing, or arresting the symptoms, clinical signs, and underlying pathology of a condition in a manner to improve or stabilize a subject's condition.

The term “topical composition” refers to a material that comprises pharmaceutically acceptable ingredients, including an active indigo agent, and is intended for administration to an animal or human subject and is applied to the surface of the skin or to the mucosa of the oral cavity, small intestine or colon, in contrast to materials that are taken orally in order to achieve systemic exposure or via intravenous (subdermal) injection. A topical composition is generally intended to have its intended effect at the site of application and does not result in significant concentrations of drug in the bloodstream or other tissues (as is the case with, for example, transdermal compositions). Topical compositions as provided herein may be administered for the purpose of alleviation of symptoms associated with a gastrointestinal disorder or dermatological diseases or conditions, for the treatment of a gastrointestinal disorder or dermatological diseases or conditions, or for prevention of a gastrointestinal disorder or dermatological diseases or conditions.

The compositions of the present disclosure can comprise, consist essentially of, or consist of, the components disclosed.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Compositions

Compositions comprised of indigo and/or an indigo derivative and an acceptable carrier are provided. In one embodiment, the composition comprises one or more of indigo, indirubin, and/or isatin. For example, the composition can be an extract that is enriched in one or more of indigo or an indigo derivative, such as indirubin and/or isatin. Alternatively, the composition is comprised of one or more of a synthetically manufactured indigo and/or an indigo derivative. In an embodiment, the composition comprises a solid amorphous dispersion of a synthetically manufactured indigo and/or an indigo derivative, such as indirubin, and a hydrophilic polymer carrier. The compositions, methods of manufacture, and in vitro analysis of exemplary formulations are now described.

The compositions described herein are comprised of a compound selected from indigo or an indigo derivative and a pharmaceutically acceptable carrier are provided. Indigo derivatives include, for example, indirubin and isatin. Some indigo derivatives exist as one or more isomers. For example, the (Z) isomer of indirubin, (Z)-2,3′-Biindoline-2′-3-dione and the (E) isomer of indirubin (Z)-2,3′-Biindoline-2′-3-dione would be considered indigo derivatives even though they may differ in biological or physical properties. Some indigo derivatives are agonists of the aryl hydrocarbon receptor (AhR). Some agonists of the aryl hydrocarbon receptor have different structures but similar, greater or lesser potency in stimulating this receptor and may behave similarly as active pharmaceutical agents for the treatment of autoimmune or autoinflammatory diseases. Known AhR ligands include endogenous compounds as in Hubbard, T. D. et al and exogenous compounds like tapinarof (also known as benvitimod or 3,5-dihydroxy-4-isopropylstilbene) and 2,3,7,8-tetrachlorodibenzo-p-dioxin (TCDD). It is believed that AhR activation results in downstream production of interleukin-22 which may mediate the activity of these compounds in the treatment of disease. Yet other compounds may induce the production of IL-22. In fact, stabilized delivery of IL-22 via linkage to polyethylene glycol (PEG) or the FC region of an antibody or other means may serve a similar biological effect in the treatment of disease.

In an embodiment, the AhR agonist compound is a prodrug of indigo or of an indigo derivative that decomposes or is metabolized into indigo and/or an indigo derivative or other AhR agonist. Examples of such prodrugs include indican, indoxyl, leuco-indigo, and the like. In one embodiment, the composition comprises indigo, an indigo derivative, a prodrug of indigo, a prodrug of an indigo derivative, and/or a combination of any of the foregoing. In another embodiment, the composition comprises indigo, an indigo derivative, a prodrug of indigo, and/or a combination thereof, exclusive of prodrugs of an indigo derivative. In another embodiment, the composition comprises an indigo derivative, a prodrug of indigo, a prodrug of an indigo derivative, and/or a combination thereof, exclusive of a prodrug of the indigo derivative indirubin.

Compositions containing indigo or a derivative, such as indirubin, that result in local but not systemic activation of the AhR are contemplated and described herein. Compositions that utilize solid amorphous dispersions of indirubin, described infra, have the advantage of being able to tune local and systemic bioavailability with a potent AhR agonist to maximize safety and efficacy in the treatment of human disease. Exemplary compositions are set forth in Example 1-10, below.

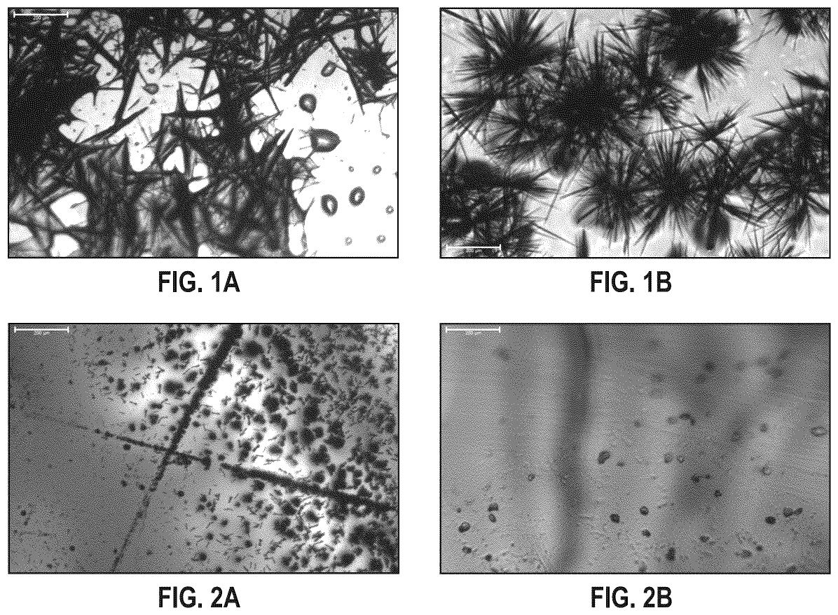

In studies, described in Examples 11-16, compositions were prepared using a synthetically manufactured indigo derivative, indirubin. As will be described, compositions of a solid amorphous dispersion of a synthetically manufactured indigo and/or an indigo derivative, such as indirubin, and a hydrophilic polymer carrier were prepared and analyzed. In an initial study, described in Example 11, synthetic crystalline indirubin was prepared. It was dissolved a suitable solvent. A solution of a hydrophilic polymeric carrier, polyvinylpyrrolidone (“PVP” or povidone) was prepared, and the drug solution and the polymer solution were combined in a weight ratio of indirubin:PVP weight ratio of 1:9. The solvents were evaporated to form a solid dispersion in which indirubin was dissolved in amorphous form. FIG. 2A shows an image of one of the solid amorphous dispersions, where a reduced amount of crystalline indirubin is apparent compared to films of FIGS. 1A-1B where synthetic indirubin is in crystalline form. FIG. 2B shows an image of another solid amorphous dispersion of indirubin and PVP, where no crystalline indirubin is observed indicating indirubin was in dissolved in the solid PVP and in the form of a solid solution.

Solid amorphous dispersions of synthetic indirubin were also prepared, as described in Example 12, from other exemplary hydrophilic polymeric carriers-butylated methacrylate copolymer, methacrylic acid-ethyl acrylate copolymer, PVP, hydroxypropyl methylcellulose acetate succinate (HPMC-AS), hydroxypropylmethylcellulose phthalate (HPMCP), polyethylene oxide, polyvinyl acetate phthalate, poloxamer P188, and polyvinyl acetate and polyvinylcaprolactame-based graft copolymer (PVAc-PVCap-PEG). The solid amorphous dispersions were observed for color and presence of crystalline indirubin. It was observed that dispersions prepared with PVAc-PVCap-PEG and with polyvinylpyrrolidone-vinyl acetate copolymer (copovidone) were purple in color and had no visibly observable crystalline indirubin, indicating indirubin was in dissolved in the solid polymer in amorphous form and in the form of a solid solution.

Example 13 describes additional studies where solid amorphous dispersions of synthetic indirubin were prepared from polyethylene oxide, HPMCP, HPMC-AS, butylated methacrylate copolymer, methacrylic acid-ethyl acrylate copolymer and PVAP using various solvents in the polymer solution. Tables 13-1 and 13-2 in Example 13 describe the visual inspection of the dispersions. The dispersions identified as samples numbers 1-3, 1-5, 1-7, and 1-12, prepared with HPMPC, HPMC-AS, methacrylic acid-ethyl acrylate copolymer, and PVAP had low crystalline content. These polymers were used to in a further study using a different drug/polymer solvent ratios while maintaining the 9:1 polymer:indirubin weight ratio. Dispersion formed were examined for the prevalence of crystalline materials and the results of these evaluations is in Table 13-2. The studies show that solid amorphous dispersions of indirubin in a hydrophilic polymer carrier form, where the indirubin is substantially in amorphous form. In an embodiment, substantially amorphous intends that the drug in the dispersion is at least about 50%, 60%, 70%, 75%, 80%, 85%, 90% or 95% amorphous, as determined, for example, by differential scanning calorimetry or x-ray diffraction, when compared to a crystalline indirubin reference sample.

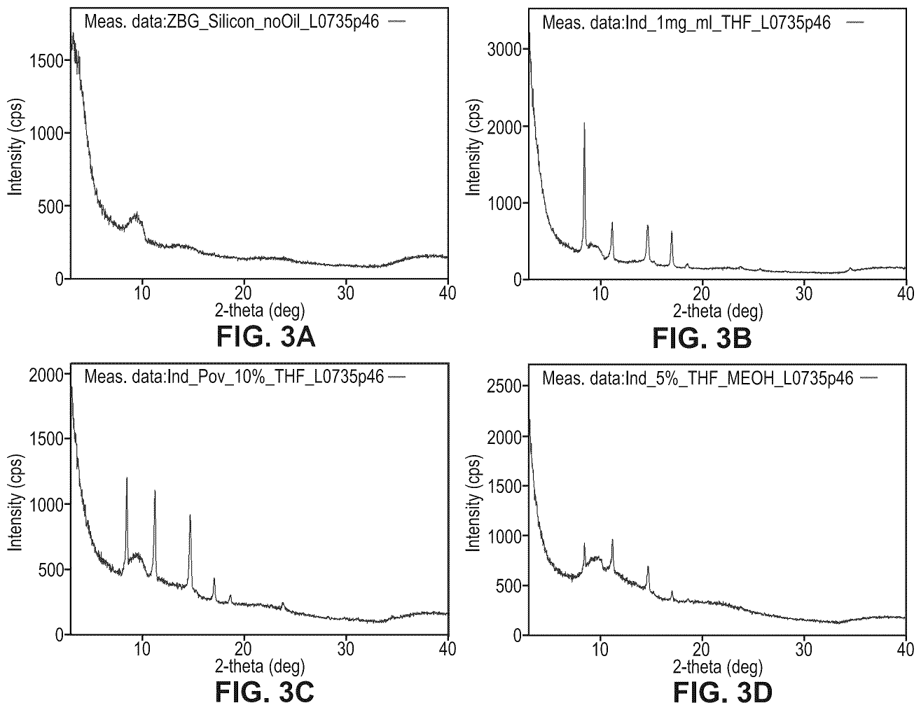

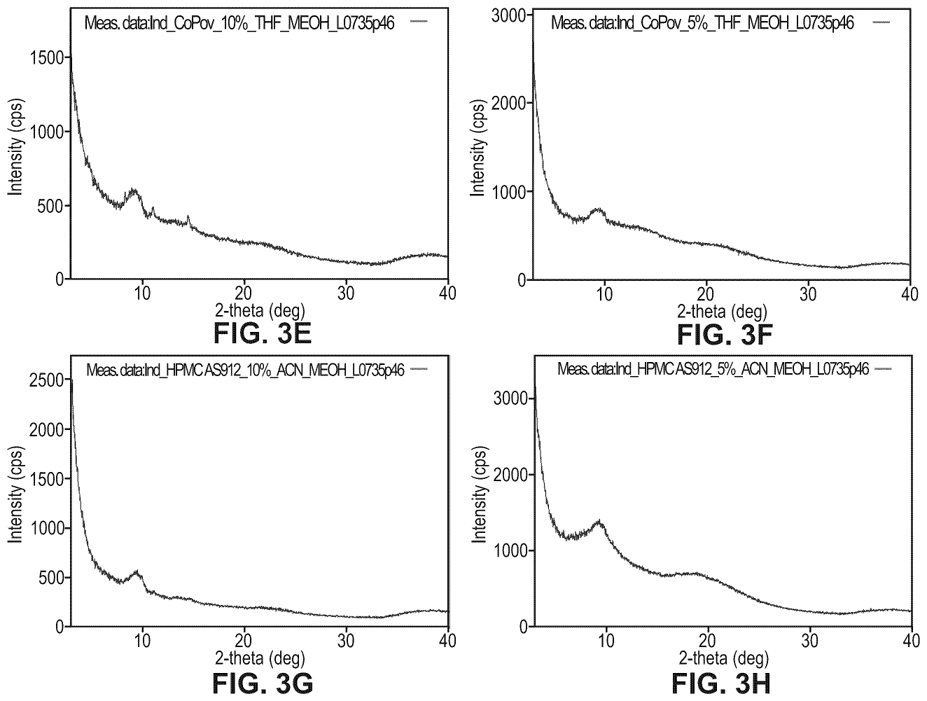

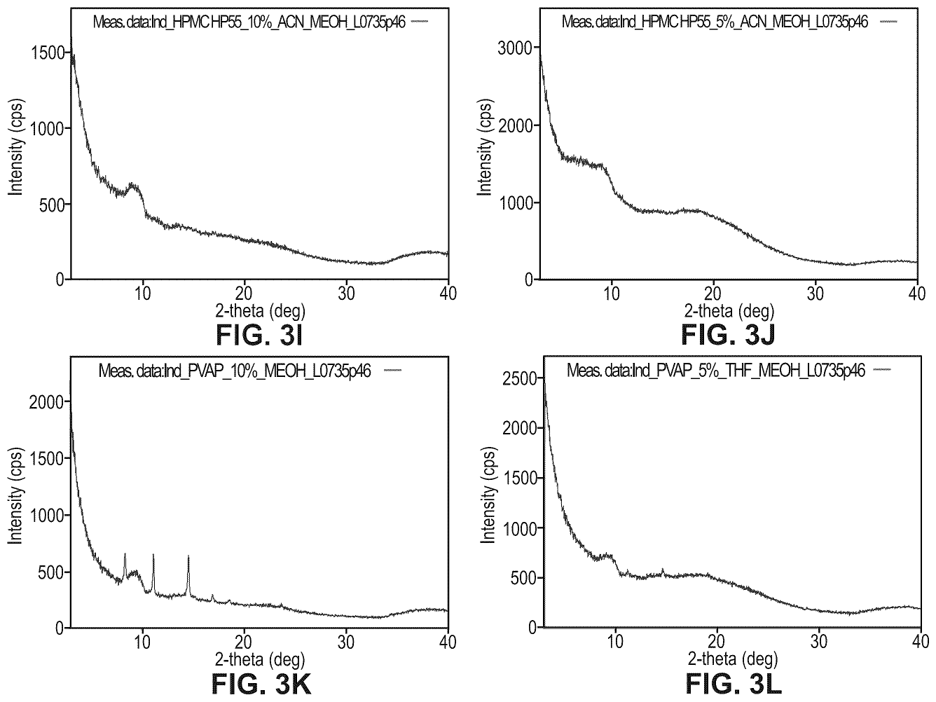

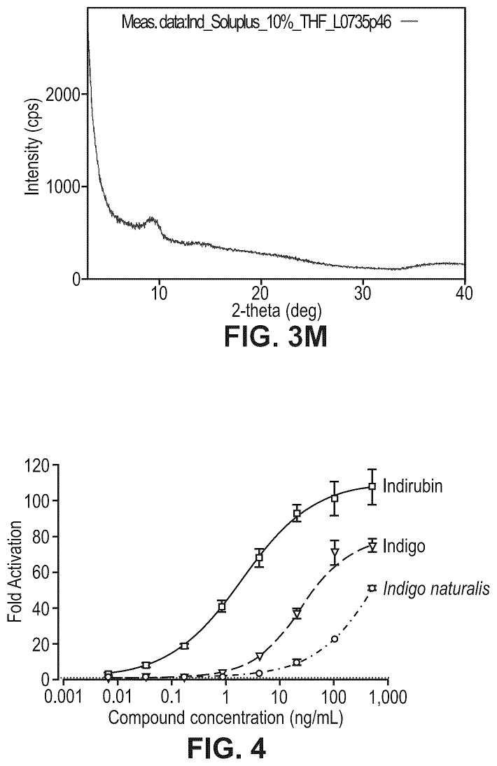

In the study of Example 14, solid amorphous dispersions with synthetic indirubin were prepared for analysis by x-ray diffraction (XRD). The dispersions were cast onto a zero background silicon sample holder slides for XRD analysis. The diffractogram for the zero background silicon sample holder is shown in FIG. 3A and for a crystalline synthetic indirubin in FIG. 3B. Solid amorphous dispersions of synthetic indirubin in PVP, PVP-AC, HPMC-AS, HPMCP, and PVAP were prepared, at 5 wt % and 10 wt % indirubin concentration, with a 9:1 indirubin:polymer weight ratio. Results are shown in FIGS. 3C-3M.

Dispersions of synthetic indirubin in PVP (povidone) at polymer:drug ratio of 9:1 with 10% indirubin (FIG. 3C) or 5% indirubin (FIG. 3D) show crystalline indirubin present in the dispersions, particularly at the higher weight percent of drug. Dispersions of synthetic indirubin in polyvinylpyrrolidone-vinyl acetate copolymer (copovidone) at polymer:drug ratio of 9:1 with 10% indirubin (FIG. 3E) or 5% indirubin (FIG. 3F) yielded dispersions with essentially no crystalline drug. That is, synthetic indirubin was in the form of a solid amorphous dispersion in the polyvinylpyrrolidone-vinyl acetate copolymer, at both 10% and 5% indirubin. Dispersions of synthetic indirubin in hydroxypropyl methylcellulose acetate succinate (HPMC-AS) at polymer:drug ratio of 9:1 with 10% indirubin (FIG. 3G) or 5% indirubin (FIG. 3H) yielded dispersions with essentially no crystalline drug. That is, synthetic indirubin was in the form of a solid amorphous dispersion in the HPMC-AS, at both 10% and 5% indirubin.

FIGS. 3I-3J are diffractograms for dispersions of synthetic indirubin in hydroxypropylmethylcellulose phthalate (HPMCP-HP55) with 10% indirubin (FIG. 3I) or 5% indirubin (FIG. 3J) yielded dispersions with the synthetic indirubin was in the form of a solid amorphous dispersion. For the dispersions prepared with polyvinyl acetate phthalate (PVAP) (FIGS. 3K-3L), synthetic indirubin was substantially in amorphous form, particularly at the 5% drug load (FIG. 3L). A dispersion of indirubin in PVAc-PVCap-PEG at a polymer:drug ratio of 9:1, shown in FIG. 3M, provided a dispersion with synthetic indirubin substantially in amorphous form.

Some of the solid dispersions displayed characteristic XRD peaks indicative of crystalline indirubin in suspension. Other dispersions displayed characteristic XRD peaks indicative of a reduced extent of crystalline indirubin in the solid amorphous dispersion (relative to the crystalline indirubin), with some indirubin in an amorphous form. See, for example, the povidone dispersions (FIGS. 3C-3D), the 10% indirubin copovidone dispersion (FIG. 3E), and the 10% PVAP dispersion (FIG. 3K). Some of the hydrophilic polymer carriers provided solid amorphous dispersions with indirubin substantially amorphous, as evidenced by a lack of XRD peaks indicative of crystalline indirubin, confirming that the indirubin was fully amorphous in suspension. See, for example, the dispersion with PVAc-PVCap-PEG (FIG. 3M), the 5% indirubin PVAP (FIG. 3L), HPMCP (FIG. 3J), HPMC-AS (FIGS. 3G-3H) and the 5% indirubin in copovidone (FIG. 3F).

Additional solid amorphous dispersions of synthetic indirubin in six different hydrophilic carrier polymers-povidone, PVAc-PVCap-PEG, (SOLUPLUS®), copovidone, HPMC-AS, HPMCP and polyvinyl acetate phthalate (PVAP)—were prepared for a further study, as described in Example 15. In this study, dispersions with indirubin to polymer ratios of 1:9 or 0.5:9.5 were prepared, and the release of indirubin into fasted-state simulated intestinal fluid was determined. For comparison, Indigo naturalis and crystalline synthetic indirubin were also tested. The amount of indirubin in fasted-state simulated intestinal fluid was determined by HPLC for samples of the fluid taken. The mean amount of indirubin in solution at each time point was determined, and is shown in Table 1 in units of μg/L and in Table 2 as a percentage, referred to a % Q, of measured amount (mass) of indirubin in solution divided by the theoretical maximum amount (mass) of indirubin in solution.

TABLE 1
Amount of indirubin in solution at each time point

| | | | | 1:9 PVAc- | 1:9 | 0.5:9.5 | 0.5:9.5 HPMC | 0.5:9.5 |
|---|---|---|---|---|---|---|---|---|
| | Indigo | Synthetic | 1:9 povidone | PVCap-PEG | Copovidone | HPMC-AS | P-HP55 | PVAP |
| Time | Naturalis | Indirubin | Dispersion | Dispersion | Dispersion | Dispersion | Dispersion | Dispersion |
| (min) | (μg/L) | (μg/L) | (μg/L) | (μg/L) | (μg/L) | (μg/L) | (μg/L) | (μg/L) |
| 5 | 1.2 | 0.02 | 17.7 | 0.99 | 8.85 | 6.28 | 21.51 | 11.52 |
| 10 | 1.6 | 0.03 | 18.0 | 1.82 | 9.40 | 10.32 | 24.29 | 13.60 |
| 20 | 2.0 | 0.03 | 17.8 | 2.60 | 10.35 | 13.29 | 24.22 | 14.14 |
| 30 | 2.3 | 0.03 | 18.2 | 3.10 | 10.62 | 13.74 | 24.18 | 14.58 |
| 60 | 2.8 | 0.04 | 17.9 | 4.11 | 11.83 | 15.11 | 23.74 | 14.48 |
| 90 | 3.0 | 0.05 | 18.0 | 4.78 | 12.56 | 15.98 | 23.41 | 14.86 |
| 120 | 3.2 | 0.07 | 18.2 | 5.13 | 13.18 | 18.13 | 23.36 | 15.32 |

TABLE 2
Amount of indirubin in % Q for each test article

| | | | | 1:9 PVAc- | | 0.5:9.5 | | |
|---|---|---|---|---|---|---|---|---|
| | | | 1:9 | PVCap- | 1:9 | sHPMC- | 0.5:9.5 | 0.5:9.5 |
| | Indigo | Synthetic | Povidone | PEG | CoPovidone | AS | HPMCP | PVAP |
| Time | Naturalis | Indirubin | Dispersion | Dispersion | Dispersion | Dispersion | Dispersion | Dispersion |
| (min) | (% Q) | (% Q) | (% Q) | (% Q) | (% Q) | (% Q) | (% Q) | (% Q) |
| 5 | 1.2 | 0.02 | 17.7 | 0.99 | 8.85 | 6.28 | 21.51 | 11.52 |
| 10 | 1.6 | 0.03 | 18.0 | 1.82 | 9.40 | 10.32 | 24.29 | 13.60 |
| 20 | 2.0 | 0.03 | 17.8 | 2.60 | 10.35 | 13.29 | 24.22 | 14.14 |
| 30 | 2.3 | 0.03 | 18.2 | 3.10 | 10.62 | 13.74 | 24.18 | 14.58 |
| 60 | 2.8 | 0.04 | 17.9 | 4.11 | 11.83 | 15.11 | 23.74 | 14.48 |
| 90 | 3.0 | 0.05 | 18.0 | 4.78 | 12.56 | 15.98 | 23.41 | 14.86 |
| 120 | 3.2 | 0.07 | 18.2 | 5.13 | 13.18 | 18.13 | 23.36 | 15.32 |

The data in Tables 1 and 2 shows that the release, or dissolution, of indirubin from the solid amorphous dispersions was far superior to indirubin in its crystalline form. Release of indirubin from the solid amorphous dispersions with povidone, HPMC-AS, HPMCP, and PVAP were between 18-24% of the total amount of indirubin in the dispersion. Less than 0.1% of the indirubin was released from the crystalline synthetic indirubin. Many of the dispersions have much better dissolution when compared to the indirubin contained within Indigo naturalis. Accordingly, embodiments contemplate compositions, including oral dosage forms and formulations, that release at least about 5%, 10%, 12%, 15%, 18%, 20%, 22% or 25% of the total amount of indirubin in the composition or in the dispersion. In other embodiments, compositions, including oral dosage forms and formulations, that release at least about 30%, 40%, 50%, 60%, 70%, 80%, 85%, 90%, 95%, 96%, 97%, 98% or 99% of the total amount of indirubin in the composition or in the dispersion are contemplated.

In another embodiment, a solid amorphous dispersion of a hydrophilic polymeric carrier (“polymer”) and indirubin (synthetic or natural) in a polymer to indirubin weight ratio of from about 5:1 to 99:1, 6:1 to 99:1, 7:1 to 99:1, 8:1 to 99:1, 10:1 to 99:1, 12:1 to 99:1, 5:1 to 75:1, 6:1 to 75:1, 7:1 to 75:1, 8:1 to 75:1, 10:1 to 75:1, 12:1 to 75:1, 5:1 to 50:1, 6:1 to 50:1, 7:1 to 50:1, 8:1 to 50:1, 10:1 to 50:1, 12:1 to 50:1, 5:1 to 30:1, 6:1 to 30:1, 7:1 to 30:1, 8:1 to 30:1, 10:1 to 30:1, 12:1 to 30:1, 7:1 to 25:1, 8:1 to 25:1, 9:1 to 25:1, 9:1 to 20:1, 10:1 to 25:1, 10:1 to 20:1, 12:1 to 25:1, 12:1 to 20:1 is provided. In one embodiment, a solid amorphous dispersion of a hydrophilic polymeric carrier (“polymer”) and indirubin (synthetic or natural) in a polymer to indirubin weight ratio of from about 6:1 to 25:1, 7:1 to 20:1, 8:1 to 20:1, greater than 9:1 and less than about 30:1, 10:1 to 30:1, 10:1 to 25:1, 10:1 to 19:1, 9:1 to 19:1, the ratio is 9:1 or 19:1, is provided.

In one embodiment, the solid dispersion comprises between about 5-80 wt % of the composition (e.g., dosage form or formulation), and in other embodiments, the solid dispersion comprises between about 6-80 wt %, 8-80 wt %, 10-80 wt %, 5-70 wt %, 8-70 wt %, 10-70 wt %, 5-60 wt %, 8-60 wt %, 10-60 wt %, 2-50 wt %, 3-50 wt %, 4-50 wt %, 5-50 wt %, 8-50 wt %, or 10-50 wt % of the composition (e.g., dosage form or formulation).

Solid amorphous dispersions can be prepared by a variety of techniques, such as the solvent evaporation approach in the working examples to form films. Other approaches for removal of the solvent(s) include spray drying, freeze drying, drum drying, and precipitation. Still other approaches use no solvent, dissolving the material directly in a polymer via techniques such as hot melt extrusion.

The synthetic indirubin manufactured for the studies here was analyzed for its carbon (¹⁴C) content. For comparison, natural indirubin was extracted and also analyzed. As described in Example 16, the percent of ¹⁴C provides a measure of the amount of carbon in the molecule originating from fossilized hydrocarbon based starting materials which are expected to have a very low ¹⁴C content in comparison to material isolated from recently living sources which would be expected to have close to an atmospheric amount of ¹⁴C. The amount of ¹⁴C in a sample changes slowly as the half-life of ¹⁴C is 5,730 years. The data is in Table 16-1 of Example 16. The synthetic indirubin has a 0.13 ¹⁴C content. In one embodiment, the synthetic indirubin of the compositions herein has a ¹⁴C content of less than about 0.9 ppt ¹⁴C, less than about 0.8 ppt ¹⁴C, less than about 0.7 ppt ¹⁴C, less than about 0.6 ppt ¹⁴C, less than about 0.5 ppt ¹⁴C, less than about 0.4 ppt ¹⁴C, less than about 0.3 ppt ¹⁴C, less than about 0.2 ppt ¹⁴C. In one embodiment, the synthetic indirubin has a ¹⁴C content of between about 0.001-1 ppt, 0.001-0.9 ppt, 0.01-1 ppt, 0.01-0.9 ppt, 0.05-1 ppt, 0.05-0.9 ppt, 0.08-1 ppt, 0.08-0.9 ppt, 0.09-1 ppt, 0.1-1 ppt, 0.1-0.9 ppt. In another embodiment, the synthetic indirubin has a fossil carbon content of greater than 10%, 20%, 30%, 40%, 50%, 60%, 70% or 80%.

In another study, in Example 17, compounds were tested for activation of the AhR receptor. The test articles were indirubin, indigo and Indigo naturalis. Each was incubated for 24 hours with reporter cells made from human Huh7 cells expressing human AhR and ARNT with luciferase expressed behind a genetic response element that responds to dimerized AhR/ARNT. The results are shown in FIG. 4 as fold activation relative to a negative control just containing the solvent DMSO as a function of concentration of the test article. Indirubin is a potent activator of the AhR receptor.

Based on the improved dissolution of amorphous indirubin provided by the dispersions described herein, a dosage form comprising a dispersion of synthetic indirubin and a hydrophilic polymeric carrier is contemplated. The dosage form, in an embodiment, is 20-fold, 30-fold, 40-fold or 50-fold more potent than Indigo naturalis (gram for gram) in its ability to activate the AhR in vivo. The dosage forms contemplated herein thus allow a dose of indirubin that is 10, 20, 30, 40 or 50 times less than the dose of Indigo naturalis. In one embodiment, the dose of indirubin on the oral dosage forms described herein is between 0.1-30 mg, 0.1-25 mg, 0.2-30 mg, 0.2-25 mg, 0.3-30 mg, 0.3-25 mg, 0.3-20 mg, 0.5-30 mg, 0.5-25 mg, or 0.5-20 mg.

In one embodiment, a formulation or an oral dosage form comprised of a solid dispersion comprising amorphous synthetic indirubin in a hydrophilic polymer carrier, wherein the indirubin is in a substantially amorphous form in the dispersion; and one or more excipients in an amount greater than about 20 wt % of the formulation and blended with the solid dispersion to form a blend. The one or more excipients is/are present in the formulation in an amount greater than about 25 wt %, 30 wt %, 35 wt %, 40 wt %, 45 wt %, 50 wt %, 55 wt %, 60 wt %, 65 wt % or 70 wt %. The excipients may include binders, disintegrants, fillers, a flavoring agent, glidants, lubricants, pH modifiers, pigments, and/or surfactants.

Binders contemplated for use include alginic acid, carbomer, carboxymethyl cellulose sodium, dextrin, ethylcellulose, gelatin, guar gum, hydrogenated vegetable oil, hydroxyethyl cellulose, hydroxypropyl cellulose, hydroxypropyl methyl cellulose, methyl cellulose, liquid glucose, maltodextrin, polymethacrylates, povidone, pregelatinized starch, sodium alginate, starch, and/or zein.

Disintegrants include, but are not limited to, calcium carboxymethyl cellulose, croscarmellose sodium, crospovidone (crosslinked polyvinyl pyrrolidone), methyl cellulose, microcrystalline cellulose, powdered cellulose, sodium starch glycolate, sodium carboxymethyl cellulose, starch, pregelatinized starch, and sodium alginate.

Fillers include sugars, such as lactose, mannitol, dextrose, sucrose, sorbitol, compressible sugar dextrates, dextran, dextrin, dextrose, maltodextrin, xylitol; celluloses such as microcrystalline cellulose, powdered cellulose; starches such as corn starch, potato starch, pregelatinized starch; mineral salts such as calcium carbonate, dibasic calcium phosphate, tribasic calcium phosphate, calcium sulfate, magnesium carbonate, magnesium oxide; and poloxamers such as polyethylene oxide.

Glidants include, for example, silicon dioxide, talc, and cornstarch.

Lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate.

pH modifiers include acids such as citric acid, acetic acid, ascorbic acid, lactic acid, tartaric acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like; and buffers generally comprising mixtures of acids and the salts of said acids.

The one or more excipients are present in the dosage form or formulation at between 20-90 wt %, 20-85 wt %, 20-80 wt %, 20-75 wt %, 20-70 wt %, 20-65 wt %, 20-60 wt %, 20-55 wt %, 20-50 wt %, 20-45 wt %, 20-40 wt %, 30-90 wt %, 30-85 wt %, 30-80 wt %, 30-75 wt %, 30-70 wt %, 30-65 wt %, 30-60 wt %, 30-55 wt %, 30-50 wt %, 30-45 wt %, 30-40 wt %, 40-90 wt %, 40-85 wt %, 40-80 wt %, 40-75 wt %, 40-70 wt %, 40-65 wt %, 40-60 wt %, 40-55 wt % or 40-50 wt %.

Based on the foregoing, it can be appreciated that the dosage form or the formulation achieves one or more of (i) the release of substantially all of the indirubin from the dosage form in the small intestine, large intestine and/or colon; and (ii) the release of substantially all indirubin from the dosage form within about 60 minutes when at a pH above 5; (iii) the release of substantially all of the indirubin from the dosage form over a period of about 8 hours when at a pH of above 5. In other embodiments, one or more of (i), (ii) or (iii) is achieved without use of an enteric polymer coating on the outside of the dosage form. In another embodiment, one or more of (i), (ii) or (iii) is achieved with the use of an enteric polymer coating on the outside of the dosage form.

The dispersion, in some embodiments, has an indirubin solubility in simulated intestinal fluid greater than a dispersion containing the same amount of indirubin in the form of crystalline indirubin, Indigo naturalis or a 1:9 povidone:indirubin solid dispersion in simulated intestinal fluid, at the same temperature. In other embodiments of the dosage form or the formulation, the indirubin remains in a substantially amorphous form the dispersion for at least about 6 months when stored at 25° C.

In other embodiments of the dosage form or the formulation, the dispersion does not contain indigo, and/or wherein the dosage form does not contain indigo. In other embodiments of the dosage form or the formulation, the hydrophilic carrier is not hypromellose acetate succinate and/or wherein the solid dispersion is not produced via precipitation from dimethylacetamide. In other embodiments of the dosage form or the formulation, the polymeric hydrophilic carrier is not povidone.

In other embodiments of the dosage form or the formulation, when administered it provides a therapeutic effect with respect to treating ulcerative colitis that is equal or superior to the therapeutic effect provided by an oral composition of Indigo naturalis containing an equivalent amount of indirubin. For example, in an embodiment, the dosage form or formulation provides a reduction in observed side effects compared to the side effects observed following administration of Indigo naturalis.

In other embodiments, a composition comprised of a compound selected from indigo, indirubin, isatin, and combinations thereof, and a pharmaceutically acceptable carrier is provided, wherein the composition provides a therapeutic effect with respect to treating UC that is substantially the same as provided by a composition of Indigo naturalis. Alternatively, the composition may provide a superior effect or superior therapeutic effect with respect to treating UC as compared to the therapeutic effect provided by a composition of Indigo naturalis. Alternatively, the composition may provide a reduction in observed side effects compared to the side effects observed following administration of Indigo naturalis. Examples of side effects observed upon oral administration of Indigo naturalis can be, for example, pulmonary arterial hypertension, liver dysfunction, headache, nausea, diarrhea, vomiting, intussusception, ischemic colitis. Alternatively, the composition provides a superior therapeutic effect with respect to treating UC as compared to the therapeutic effect provided by a composition of the carrier and indigo, indirubin, or isatin individually. The effects of the composition when compared to Indigo naturalis, in one embodiment, are ascertained when the compositions are administered via the same route of administration and/or at essentially the same dose of at least one of the active compounds, e.g., indigo or indirubin or another indigo derivative.

The compound or compounds in the composition, e.g., indigo, indirubin, and/or isatin, is/are synthetically produced or synthetically synthesized, in one embodiment. In another embodiment, the compound or compounds in the composition, e.g., indigo, indirubin, and/or isatin, is/are isolated from a plant extract and/or resulting from processing of a plant extract to obtain a refined extract that consists of the one or more compound or compounds. That is, the processing technique removes from the native or raw extract essentially all other compounds other than the specifically desired compounds. The compound or compounds, whether synthetically produced or obtained from a processing technique of a plant extract, is/are combined with a pharmaceutically acceptable carrier to provide a composition for administration to a subject.

In one embodiment, a composition that is not Indigo naturalis and that is comprised of indigo and/or an indigo derivative is contemplated. In these compositions, indigo and/or the indigo derivative may be synthetically produced, extracted and/or isolated from plants, or made via fermentation or bioreactor. Chemical synthesis and biosynthesis of indigo and/or indigo derivatives is described, for example, in Ensley et al., Science, 167 (1983)). Processing of plants to enrich the extract in one or more of indigo and/or an indigo derivative is another approach to production of a composition, that is not Indigo naturalis, and that comprised of indigo and/or an indigo derivative.

Compositions comprising a single compound, in some embodiments, comprise between about 0.001-15 wt %, 0.001-10 wt %, 0.001-5 wt %, 0.01-15 wt %, 0.01-10 wt %, 0.01-5 wt %, 0.05-15 wt %, 0.05-10 wt %, 0.05-5 wt %, 0.1-15 wt %, 0.1-10 wt %, 0.1-5 wt %, 0.2-15 wt %, 0.2-10 wt %, 0.2-5 wt %, 0.3-15 wt %, 0.3-10 wt %, 0.3-5 wt %, 0.4-15 wt %, 0.4-10 wt %, 0.4-5 wt %, 0.5-15 wt %, 0.5-10 wt %, 0.5-5 wt %, 0.6-15 wt %, 0.6-10 wt %, 0.6-5 wt %, 0.7-15 wt %, 0.7-10 wt %, 0.7-5 wt %, 0.8-15 wt %, 0.8-10 wt %, 0.8-5 wt %, 0.9-15 wt %, 0.9-10 wt %, 0.9-5 wt %, 1-15 wt %, 1-10 wt %, or 1-5 wt % of the single compound based on the total weight of the composition. The therapeutic compound, can be indigo or an indigo derivative.

In other embodiments, the composition comprises at least two compounds selected from indigo, indirubin, and/or isatin. The compounds are present, in some embodiments, in one of the following weight percentages based on the weight of the active agent (therapeutic) compounds in the composition (e.g., not based on total weight of the composition):
(i) between about 55-95% indirubin, 5-45% indigo, and 0-40% isatin;
(ii) between about 40-80% indirubin, 20-60% indigo, and 0-40% isatin;
(iii) between about 20-60% indirubin, 40-80% indigo, and 0-40% isatin;
(iv) between about 0-40% indirubin, 55-95% indigo, and 5-45% isatin;
(v) between about 0-40% indirubin, 40-80% indigo, and 20-60% isatin;
(vi) between about 0-40% indirubin, 20-60% indigo, and 40-80% isatin;
(vii) between about 5-45% indirubin, 0-40% indigo, and 55-95% isatin;
(viii) between about 20-60% indirubin, 0-40% indigo, and 40-80% isatin; or
(ix) between about 40-80% indirubin, 0-40% indigo, and 20-60% isatin.

In some compositions comprised of indigo, indirubin, and/or isatin at least one of the compounds is present in the composition in an amount at least 10%, 15%, 20%, or 25% greater by weight than the amount by weight in Indigo naturalis or in an amount at least 10%, 15%, 20%, or 25% greater relative to the amount present in Indigo naturalis. In some compositions comprised of indigo, indirubin, and/or isatin at least one of the compounds is present in the composition at least one of the compounds is present in the composition in an amount at least 10%, 15%, 20%, or 25% less by weight than the amount by weight in Indigo naturalis or in an amount at least about 10%, 15%, 20%, or 25% less relative to the amount present in Indigo naturalis.

In one embodiment, the active agent in the compositions comprises, consists essentially of, or consists of indirubin and indigo; or indirubin and isatin; or indigo and isatin. When indirubin and indigo are present in the composition, they may be in a ratio of indirubin to indigo from about 1:100 to 10:1, 1:30 to 30:1, 1:25 to 25:1, 1:20 to 20:1, 1:15 to 15:1, 1:10 to 10:1. When indirubin and isatin are present in the composition, they may be in a ratio of indirubin to isatin from about 1:30 to 30:1, 1:25 to 25:1, 1:20 to 20:1, 1:15 to 15:1, 1:10 to 10:1. When indigo and isatin are present in the composition, they may be in a ratio of indigo to isatin from about 1:30 to 30:1, 1:25 to 25:1, 1:20 to 20:1, 1:15 to 15:1, 1:10 to 10:1. Alternatively, the various combinations of active agents can be in a ratio of the first active agent to the second active agent from about from about 100:1 to 1:10, 10:1 to 50:1, 15:1 to 50:1, 20:1 to 50:1, 25:1 to 50:1, 15:1 to 45:1, 20:1 to 45:1, 25:1 to 45:1, 30:1 to 45:1, 20:1 to 30:1, 20:1 to 35:1, 20:1 to 40:1, or 30:1 to 35:1. In another embodiment, the ratio of first active agent to the second active agent is about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1 or 100:1.

In another embodiment, the composition comprises at least two compounds selected from indigo, indirubin, and isatin, and a pharmaceutically acceptable carrier, wherein the composition is not Indigo naturalis. In some embodiments, the weight percent of the first active agent, based on the total weight of the active agents in the composition, is between about 25-99 wt %, 25-98 wt %, 25-96 wt %, 25-95 wt %, 25-90 wt %, 25-85 wt %, 25-75 wt %, 25-70 wt %, 25-65 wt %, 25-60 wt %, 25-55 wt %, 25-50 wt %, 25-45 wt %, 25-40 wt %, or 25-35 wt %. In other embodiments, the weight percent of the second active agent, based on the total weight of the active agents in the composition, is between about 25-99 wt %, 25-98 wt %, 25-96 wt %, 25-95 wt %, 25-90 wt %, 25-85 wt %, 25-75 wt %, 25-70 wt %, 25-65 wt %, 25-60 wt %, 25-55 wt %, 25-50 wt %, 25-45 wt %, 25-40 wt %, or 25-35 wt %.

In any of the compositions described herein, indigo and indigo derivatives may be substituted with boron, H3 or radioactive moieties.

In any of the compositions described herein, indigo and/or the indigo derivative can be in the form of a solid. This solid may be highly crystalline or bound to inorganic and organic material that has crystalized out of solution or been evaporated down into a solid. This material may be processed into a powder. The powder may have a uniform or widely distributed average particle size. The particle size may be on the nanometer scale, the micrometer scale or larger. Powders of uniform particle sizes may have different solubility or bioavailability based on their crystalline structure, surface area, co-crystals or isoform. This relative bioavailability or lack thereof may make these agents more effective, less effective or safer or less safe in treated patients with a disease. In particular, larger particle sizes or reduced surface area may be less soluble and, therefore, less bioavailable resulting in less systemic exposure and therefore better safety while maintain local activity and efficacy. In particular, treating patients with formulations made from these particles may result in reduced liver toxicity, reduced serotonin levels or reduced pressure in the lungs or a reduced chance of developing pulmonary arterial hypertension compared to more bioavailable particles such as those that may be found in Indigo naturalis.

The solid particles of indigo and/or indigo derivatives can have average particle sizes of less than about 5000 nm, less than 3000 nm, less than 2000 nm or less than 1500 nm. In other embodiments, the solid particles of indigo and/or indigo derivatives can have average particle sizes of equal to or greater than about 1500 nm, 2000 nm, 2500 nm, 3000 nm or 5000 nm. In some embodiments, more than about 60%, 70%, 75%, 80% 85% or 90% of the particles are larger than about 1500 nm, 2000 nm, 2500 nm, 3000 nm or 5000 nm. In some embodiments, more than about 60%, 70%, 75%, 80% 85% or 90% of the particles are smaller than about 1500 nm, 2000 nm, 2500 nm, 3000 nm or 5000 nm. In some embodiments, the particles can contain optionally include a surface stabilizer, or may exclude a surface stabilizer.

In some embodiments, the indigo and/or indigo derivatives can be bound to or mixed with particles that contain calcium carbonate, cellulose, organic resins, inorganic resins, clay or zeolites. In some embodiments, the indigo and indigo derivatives can be free from calcium carbonate or cellulose.

In some embodiments, the solid powder contains multiple active agents, such as indigo and/or one or more indigo derivatives, in the same particle or in the same population of particles. In some embodiments, the solid powder contains a mixture of distinct particles each comprising an active agent of indigo or a particular indigo derivative. In some embodiments the particles are at least about 95%, 96%, 97%, 98% or 99% pure or are over 99.9% pure. The solid particles can each have the same average particle size or can have different average particles sizes. In other embodiments, the solid particles may have the same solubility in a selected solvent (e.g., water or ethanol) or may have the same bioavailability.

In some embodiments, the composition differs in solubility or bioavailability from Indigo naturalis that contains the same about of a given active agent (e.g., indigo or an indigo derivative). This difference in solubility may be seen in solvents including water, low pH water, or certain formulation solvents, such as ethanol, n-methyl pyrrolidone, polyethylene glycol, or dimethylsulfoxide. This difference in solubility or bioavailability may be to the particle surface area, particle size, particle shape, contaminants, purity and/or hydrophobicity of the particles or excipients included in a given formulation.

The indigo and indigo derivatives may be free from or reduced substantially of impurity found in Indigo naturalis, in some embodiments. In some instances, the impurity may be an allergen or toxin. In some embodiments the impurity may be indigo, indirubin, isatin, indospicine or 3-nitropropionate. In some embodiments the impurity may be inorganic, or may be a heavy metal, such as arsenic, cadmium, lead or mercury. The impurity may also be a pesticide, silicon dioxide, limestone, cellulose, water, or calcium carbonate. In one embodiment, the indio and/or indigo derivative is substantially free of one or more of such impurities.

It is also contemplated that the indigo and/or indigo derivatives may have less bioburden per gram than Indigo naturalis, in some embodiments. For example, the indigo and/or indigo derivatives may have a reduced bacterial count, reduced yeast count, reduced mold count, reduced E-coli count and/or reduced salmonella count than a preparation of Indigo naturalis. In some embodiments the indigo and/or indigo derivatives may be more resistant to the growth of microorganisms. In some embodiments, the indigo and/or indigo derivatives may have less than about 15,000, 25,000, 35,000 or 50,000 colony forming units per gram. In some embodiments, the indigo and/or indigo derivatives may be sterile. In some embodiments, the indigo and/or indigo derivatives may include a preservative that inhibits bacterial and/or microbe growth over time.

The indigo and/or indigo derivatives may differ in color than Indigo naturalis and may be more dark blue, light blue or blackish. In some embodiments, the indigo and/or indigo derivatives may be purplish, reddish, orangish, pinkish, or brownish. In some embodiments, the indigo be lighter in color, white or clear. The indigo and/or indigo derivatives may be reduced into their reduced forms changing their color or rendering them less colored or even colorless or white. In some embodiments, the indigo and/or indigo derivatives may have particle sizes in the nanometer range or may have a unique structure that alters their color compared to what is found in Indigo naturalis or what has been purified from synthetic preparations.

Compositions containing Indigo naturalis may change the color of the stool, urine or other bodily fluids or surfaces. This may not be desirable to a patient being treated. Additional dyes or colorants may be added to a formulation containing Indigo naturalis or Indigo and/or indigo derivatives in order to normalize the color of the stool urine or bodily fluids. In one example, a reddish dye such beetroot extract or FD&C red no. 3 may be added to Indigo naturalis in order to keep stools brown in color rather than turning blueish or greenish.

Indigo and/or indigo derivatives, in one embodiment, have a half maximal effective concentration (EC₅₀) of less than about 10 nM, 8 nM, 6 nM, 5 nM, 4 nM, 3 nM, 2 nM, 1 nM, 0.5 nM, 0.2 nM or 0.1 nM for the human aryl hydrocarbon receptor as measured via a yeast aryl hydrocarbon receptor using human aryl hydrocarbon receptor and aryl hydrocarbon nuclear transporter proteins with a readout in beta-galactosidase activity or luciferase activity.

In some instances the half maximal effective concentration (EC₅₀) of two of indigo and/or indigo derivatives on the activation of the human aryl hydrocarbon receptor would be reduced in an additive manner or in a synergistic mechanism to be a greater reduction than what is anticipated from the additive response of the single indigo and/or indigo derivatives.

Indigo and indigo derivatives are exemplary kinase and transcription factor inhibitors. The compositions mentioned above can comprise agents that also inhibit a kinase or transcription factor. These kinase or transcription factors may be in a post-translationally modified or unmodified form including but not limited to being in a phosphorylated or unphosphorylated form. They may be free or associate with other enzymes or cofactors as part of a complex. In one embodiment the agents can be an inhibitor of a signal transducer and activator of transcriptions (STAT) such as STAT3, a Janus kinase (JAK) such as JAK2, a mitogen-activated protein kinase (MAPK) which includes extracellular signal-regulated kinase (ERK) such as ERK2, a tyrosine-protein kinase such as C-src tyrosine kinase, a casein kinase such as casein kinase 1, a cyclin-dependent kinase (CDK) such as CDK1, CDK2 or CDK5 or a glycogen synthase kinase (GSK) such as GSK3B at a half maximal effective concentration (IC₅₀) of less than about 100 μM, 50 μM, 25 μM, 10 μM, 5 μm or 1 μM.

Indigo and/or indigo derivatives may also be cytotoxic to fast dividing cells. The compositions mentioned herein can comprise agents that are cytotoxic to fast diving cell such as CD4+ T-cells at a half maximal effective concentration (IC₅₀) of less than about 100 μM, 50 μM, 25 μM, 10 μM, 5 μm or 1 μM.

As mentioned, the compositions also comprise a carrier or a pharmaceutically acceptable vehicle or excipient, to ease administration to a person in need. In one embodiment, the composition is formulated for localized delivery. Localized delivery includes local delivery to a tissue, such as skin (e.g. via topical delivery), to an organ, or to the gastro-intestinal tract (e.g., via oral delivery, rectal delivery). In one embodiment, localized delivery is to intact or disrupted mucosal tissue. In another embodiment, localized delivery is to the oral mucosa. In one embodiment, the localized delivery is to the small intestine, the esophagus, stomach, oral mucosa, rectum or to the colon. A skilled artisan will appreciate that the carrier will vary depending on the desired composition, which can take a number of different forms, including, for example, a tablet, a capsule, a gel cap, an enteric-coated tablet, an enteric-coated tablet capsule, an enteric-coated gel cap, a collection of microspheres, a collection of nanoparticles, a suspension, a powder for suspension, an orally-disintegrating tablet, a buccal tablet, an orally-dissolving film, a lozenge, a suppository, foam, an enema, an ointment, a cream, a lotion, a gel, and the like. Information regarding suitable formulations is found, for example, in “Remington: The Science and Practice of Pharmacology,” 22nd edition, (Pharmaceutical Press, 2013).

In one embodiment, the composition comprises one or more carriers to achieve colonic delivery of the compound or compounds. A composition designed for colonic release may employ one of the following features to target release of the compounds predominantly in the colon relative to other portions of the gastrointestinal tract: (1) the generally increasing pH profile of the lumenal contents up to the ileocecal junction; (2) the relatively constant small intestinal transit time of an orally administered composition; and (3) the presence of endogenous bacteria in the colon (M. Ashford et al., J. Drug Targeting, 2:241-258 (1994)).

Oral dosage forms formulated for localized delivery in the colon, in one embodiment, comprise a carrier in the form of a pH-sensitive polymer. The pH varies along the length of the gastrointestinal tract, from a pH of 1 to 2 in the stomach and to a pH of around 7.5 in the distal small intestine. The pH then declines from the end of the small intestine to the colon and gradually increases once again in the colon. Accordingly, a composition comprising a carrier that is pH-sensitive, for example, a pH-sensitive coating membrane or a pH sensitive polymeric hydrophilic carrier, can achieve release of the compounds in the colon. A pH-sensitive polymer that breaks down at a basic pH and remains intact at acidic pHs protects the compound from the acidic pH of the stomach and proximal small intestine. These polymers then break down in the more basic pH of the terminal ileum, thus providing a targeted drug delivery to the colon. Polymers based on poly-methacrylate such as Eudragit® L and Eudragit® S have frequently been used for this purpose, and each one has its own unique pH value at which it dissolves. These two polymers have been mixed in different ratios to form a coating with an optimized dissolution rate. Additionally, coatings with these polymers are designed to be relatively thick to prolong their dissolution, and provide a controlled or an extended drug release. In one embodiment, the polymeric hydrophilic carrier is a pH sensitive polymer that dissolves or becomes soluble at a pH between 4.5-7.5 in the intestinal tract or in simulated gastric fluid at a temperature of 25° C. or 30° C. or 35° C. In another embodiment, the polymeric hydrophilic carrier dissolves or becomes soluble at a pH between 4.5-7.5 and is insoluble at pH 1.5-3.5 in the intestinal tract or in simulated gastric fluid at a temperature of 25° C. or 30° C. or 35° C.

In one embodiment, the composition comprises a carrier that achieves a delayed release of the indigo compound(s) and/or indigo derivative compound(s) for a period of time approximately corresponding to the residence time in the small intestine. An exemplary composition is one having a single enteric polymer layer or multiple enteric polymer coating layers. Compositions with multiple enteric polymer coating layers may comprise an outermost layer consists of an enteric polymer that begins to dissolve at pH 6.8 to 7.2 and an inner coating layer of an enteric polymer that begin to dissolve at pH 5.0 to 6.3 in an amount such complete dissolution substantially occurs within the proximal colon. Thus, the function of the outermost coating layer is to prevent release of the therapeutic agent as the dosage form transits the gastrointestinal tract to the distal small intestine, and the function of the inner coating layers is to further delay release of the therapeutic agent until the dosage form has reached the proximal colon. Examples of such compositions are detailed in EP0825854.

Exemplary pH sensitive polymers include cellulose acetate phthalate; cellulose acetate trimelliate; hydroxypropyl methylcellulose phthalate; hydroxypropyl methylcellulose acetate succinate; polyvinyl acetate phthalate; hypromellose acetate succinate; hypromellose phthalate; poly(methacrylic acid, methyl methacrylate) 1:1; poly(methacrylic acid, ethyl acrylate) 1:1, poly(methacrylic acid, methyl methacrylate) 1:2, and a mixture of poly(methacrylic acid, methyl methacrylate) 1:1 and poly(methacrylic acid, methyl methacrylate) 1:2 in a ratio of 1:10 to 1:2. These pH sensitive polymers may be made in different grades which allow for dissolution at various pHs. For example, HPMC is available in different grades including HPMCAS F912, HPMCAS 716 and HPMCAS 126 that vary in their relative amount of monisuccinic acid and acetic acid esters giving them unique stability and dissolution properties at various pHs.

Alternatively, these pH sensitive polymers may also be incorporated into the dosage forms themselves. In one example the polymer can be used to dissolve the AhR agonist and may result in the form of a solid suspension. In another example the AhR agonist may be indirubin. In another example the AhR agonist may be a partially or fully amorphous solid suspension when dissolved within one of these pH sensitive polymers. In one example, this will protect the AhR agonist from both being degraded by the low pH of the stomach and may limit systemic adsorption. When in the form of a solid dispersion made of a hydrophilic polymer, including a pH sensitive polymer, the drug product offers improved better dispersion and dissolution when dosed orally than a dosage from that is merely coated with the same hydrophilic polymer or pH sensitive polymer. These solid suspensions dissolved in a hydrophilic polymer or a pH sensitive polymer may be in the form of a tablet or capsule which is subsequently coated with a single enteric polymer layer or multiple enteric polymer coating layers that may be of the same of different compositions as the polymer used in the solid suspension.

Colonic targeted delivery can also be achieved by relying on the aerobic and anaerobic microorganisms like Escherichia and Clostridium species, respectively, resident in the colon. These bacteria contain several hydrolytic and reductive metabolizing enzymes that can catalyze a range of reactions. Polysaccharides such as chitosan, guar gum, pectin, etc., can be employed as release rate-controlling components in colon-targeted dosage forms as these polysaccharides are resistant to gastric and intestinal enzymes, but are metabolized by anaerobic bacteria in the colon. Modification of polymers tailored for enzymatic degradation in the colon is another approach, and acetyl derivatives of guar gum, azo-aromatic polymers, are examples of polymers susceptible to degradation by colonic enzymes. These polymers can be used to coat the indigo and/or indigo derivative to protect them from degradation by peptidases in the stomach and small intestine while still permitting drug release in the colon.

Another approach towards colon-targeted drug delivery includes embedding the drug in polymer matrices to trap it and release it in the colon. These matrices can be pH-sensitive or biodegradable. Timed-released formulations are based on the drug being released in the colon after a specified amount of time. This approach is dependent on the transit time through the small intestine, which is and known to vary between 3 and 4 hours. A combination of pH-sensitive polymers and a timed-release approach to achieve colon-specific delivery may also be used, such as enclosing a core of the indigo or indigo derivate compound(s) in polymeric layers of a hydrophilic layer sandwiched between two pH-sensitive layers.

Bioadhesive compositions are another approach for localized delivery. A bioadhesive composition allows a formulation to remain in contact within an organ, in this case the colon, for a long period of time. Some of the polymers which have been explored as bioadhesive components for these systems include polycarbophils, polyurethanes, and polyethylene oxide.

Multiparticulate systems have a smaller particle size compared to single-unit systems, and studies have shown that they can reach the colon more quickly since they pass through the GI tract more easily. Microspheres are one example of a multiparticulate system that can be loaded with a drug for colonic delivery. An example is a core of the indigo or indigo derivate compound(s) mixed with cross-linked chitosanor a polysaccharide pectin to form microspheres that are then coated with a pH-sensitive polymer available under the tradename EUDRAGIT®.

Polysaccharide-based carriers are another option to achieve colon-specific delivery of the indigo or indigo derivate compound(s). Examples include xanthan gum and guar gum formulated with the compound(s) to form solid matrix tablets. A combination of polysaccharides as the carrier is also effective for achieving colon-specific delivery. Cellulose derivatives may be used alone or in combination with a polysaccharide, as cellulose is not absorbed systemically when administered orally. Non-enteric cellulose esters such as cellulose acetate are insoluble in water and their solubility is independent of pH. These can be used in insoluble, permeable coatings. Enteric cellulose esters such as cellulose acetate phthalate (CAP) and hydroxypropyl methylcellulose phthalate (HPMCP) have solubilities which are pH-dependent. They are insoluble in highly acidic conditions, but when the pH reaches a certain range they dissolve. The pH at which the polymer dissolves varies depending on the extent of esterification. Some examples of carbohydrate mixtures include pectin-HPMC, chitosan-HPMC, chitosan-pectin, guar gum-chitosan, and dextran-chitosan.

Additional embodiments may include single carriers or a combination of carriers both to prevent the indigo compound(s) and/or indigo derivative compound(s) from being released in the stomach and to maximize or minimize absorption, retention or dispersion or solubility across or through certain anatomical regions. Examples include moieties that become adhesive to areas of inflammation, specific cell types or mucosa, moieties that prolong the bioavailability either by swelling or gelling, moieties that increase local or systemic penetration into or through the lumen of the small intestine or colon, moieties that enhance or prevent the disposition of the active agents and moieties that increase the solubility when exposed to changes in the colon. Some of the moieties may be activated by pH, time or enzymatic activity specific to the small intestine or colon.

In some embodiments, carriers to target inflammatory lesions may include hydrogels, polymers or fibers made of ascorbyl palmitate.

Some embodiments may include carriers to increase solubility or stability. Exemplary carriers include surfactants such as tween-80, and complexing-agents such as cyclodextrins and co-solvents such as pentanol.

The compositions described herein may also contain relatively small amounts, e.g., less than about 10% (w/w) of one or more auxiliary excipients including but not limited to pH modifying agents, preservatives, thickening agents, gel-forming agents, emulsifying agents, antioxidants, scent agents, and the like. Compounds suitable for incorporation may be found, e.g., in R. C. Rowe, et al., Handbook of Pharmaceutical Excipients (4^{th} Ed.), Pharmaceutical Press, London, 2003.

The compositions described herein may also comprise an agent to alter viscosity or to gel the composition. Examples include cellulose ethers such as hydroxypropyl cellulose, hydroxypropyl methylcellulose, carboxymethyl cellulose, sodium carboxymethyl cellulose, hydroxyethyl cellulose, and the like; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like, and methacrylates such as those available under the tradename Eudragit® from Rohm Pharma. Other gelling agents include polyoxyethylene-polyoxypropylene copolymers (poloxamers) such as those available under the tradename “Lutrol®”, and the like. Other gelling agents are those absent free carboxyl groups such as, for instance, hydroxypropylcellulose, hydroxypropylmethylcellulose, hydroxyethylcellulose, methylcellulose, organo/cold water soluble cellulose, hydroxyethylmethylcellulose, ethylcellulose, ethyl(hydroxyethyl)cellulose.

The compositions described herein may also contain an antioxidant. The amount of antioxidant, if present, will typically range from about 0.005% to about 3.0% by weight of the composition. Illustrative ranges include from about 0.01% to about 2.5% by weight antioxidant, from about 0.05% to about 2% by weight antioxidant, and from about 0.1% to about 1.5% by weight anti-oxidant. Suitable antioxidants include, for example, butylated hydroxyanisole (BHA), butylated hydroxytoluene (BHT), tertiary butyl hydroquinone, propyl gallate, α-tocopherol, sodium metabisulfite, and the like.

The compositions described herein may further contain one or more preservatives in an amount typically ranging from about 0.01% to about 2.0% by weight of the composition. Illustrative preservatives include, for example, phenoxyethanol, methyl paraben, propyl paraben, butyl paraben, benzyl alcohol, and the like.

A wide variety of methods may be used for preparing the compositions described above. Broadly speaking, the compositions may be prepared by combining together the components at a temperature and for a time sufficient to provide a pharmaceutically effective and elegant composition. The term “combining together”, as used herein, means that all of the components of the compositions may be combined and mixed together at about the same time. The term “combining together” also means that the various components may be combined in one or more sequences to provide the desired product. The formulation can be prepared on a weight/weight (w/w) or a weight/volume (w/v) basis depending upon the form of the final dosage form.

The compositions may be packaged for use in a medical setting or for retail distribution directly to the consumer (i.e., an article of manufacture or kit). Such articles will be labeled and packaged in a manner advising the patient how to use the product for therapy. Such instructions will include the duration of treatment, dosing schedule, precautions, dietary recommendations or foods or medications to avoid etc. These instructions may be in the form of pictures, written instructions, or a combination thereof. They may be printed on the side of the packaging, be an insert, or any other form of communication appropriate for the retail market.

The composition may conveniently be presented in dosage unit form and may be prepared by any of the methods well known in the art of pharmacy. For example, the composition can be placed in an appropriate container, such as a tamper-proof bottle, a foil-lined blister pack, a time-released jar, or a device for dispensing unit dosages of the formulation, such as a bottle or dropper that dispenses a controlled pre-determined dosage of the composition.

The methods and compositions can optionally be used in combination with one or more other therapies, such as one or more therapeutic agents, surgery and/or radiation. In other embodiments, the methods and compositions provided herein are used in combination with one or more therapeutic agents.

### Methods of Treatment

Methods for treatment using any of the compositions (e.g., the dosage form or formulations) described herein are contemplated. In one aspect, methods for treating are related to subject with gastro-intestinal inflammatory diseases, such as ulcerative colitis or Crohn's disease.

Accordingly, a method for treating ulcerative colitis (UC) is provided. The method comprises administering to a subject a therapeutically effective amount of a composition comprising indigo, indirubin, isatin, or a combination thereof, wherein the composition is not Indigo naturalis, whereby the administering treats UC in the subject.

In one embodiment, the subject with UC is refractory or intolerant to treatment with a known therapy, including but not limited to, 5-aminosalicylic acid, a prodrug of 5-aminosalicylic acid, a steroid, an anti-TNF-alpha antibody, a Janus kinase inhibitor (e.g., peficitinib, tofacitinib), an integrin receptor antagonist, such as vedolizumab (an alpha-4B7 integrin antagonist), a sphingosine-1-phosphate receptor modulator, such as ozanimod, and/or any other known drug therapy for UC (e.g., Ko, C. et al., Gastroenterology, 156(3):748-764 (2019)).

In another embodiment, the subject with UC has previously been treated with 5-aminosalicylic acid or a prodrug of 5-aminosalicylic acid, such as sulfasalazine.

In another embodiment, the subject has mild to moderate UC. In another embodiment, the subject has moderate to severe UC. Mild to moderate UC is defined by the American Gastroenterological Association as fewer than 4-6 bowel movements per day, mild or moderate rectal bleeding, absence of constitutional symptoms, low overall inflammatory burden, and absence of features suggestive of high inflammatory activity (Ko, C. et al., Gastroenterology, 156(3):748-764 (2019)).

Accordingly, a method for treating ulcerative colitis (UC), for treating inflammatory bowel disease, for preventing recurrence of UC, for treating UC in a subject previously treated with 5-aminosalicylic acid or a prodrug of 5-aminosalicylic acid, for preventing recurrence of UC, for treating UC in a subject refractory or intolerant to treatment with a known therapy are each contemplated, and comprise administering the a subject in need a dosage form or formulation (a composition) as described herein, to provide a therapeutically effective amount of the aryl hydrocarbon receptor agonist, such as indirubin.

More generally, a method for treating inflammatory bowel disease or for treating a gastro-intestinal inflammatory disease in a subject is provided. The method comprises administering to a subject a therapeutically effective amount of a composition comprising indigo, indirubin, isatin, or a combination thereof, wherein the composition is not Indigo naturalis, whereby the administering treats the inflammatory bowel disease in the subject. In one embodiment, the inflammatory bowel disease is not Crohn's disease.

In one embodiment, the amount of drug compound needed to achieve and or maintain a clinical response after 8 weeks of treatment is less than 0.5 grams per day of Indigo naturalis.

In one embodiment, treating is for a duration of longer than about 8 weeks or about 10 weeks to achieve and/or maintain a clinical response and/or clinical remission.

In another embodiment, treating or administering of the indigo and/or indigo derivative is for maintenance of clinical remission.

In another embodiment, a ratio of indigo to indirubin that is greater than about 3-fold is administered in the composition to achieve and/or maintain clinical response after about 8 weeks of treatment.

In another embodiment, a dose of greater than about 6 mg of indigo is administered to achieve clinical response and/or maintain clinical remission.

In another embodiment, a dose of greater than about 2 mg of indigo is administered daily for 8 weeks to achieve clinical response and/or maintain clinical remission.

In yet another embodiment, a dose of greater than about 2 mg of indirubin is administered daily for 8 weeks to achieve clinical response and/or maintain clinical remission.

In still another embodiment, a dose of less than about 0.66 mg of indirubin is administered daily for 8 weeks to achieve clinical response and/or maintain clinical remission.

In another embodiment, it is contemplated to administer a composition of Indigo naturalis where indigo is present in the composition at a concentration greater than 0.3 wt % and/or indirubin is present in the composition at a concentration greater than 0.1 wt % to achieve and/or maintain a clinical response.

In one embodiment, the dosage form or formulation yields a non-therapeutic blood level of indirubin. In one embodiment, the dosage form or formulation yields a systemic blood level of less than about 1 nM of the compound.

In some embodiments, the clinical response is a single measurement or, in other embodiments, is a compilation of measurements, at or before or after 8 weeks of treatment, where the measurement(s) includes but is/are not limited to: clinical response on the Mayo score defined as a decrease from baseline in Mayo score of ≥3 points and ≥30%, with an accompanying decrease in the subscore for rectal bleeding of ≥1 point or absolute subscore for rectal bleeding of 0 or 1, clinical remission defined as a Mayo score ≤2 with no individual subscore >1) and rectal bleeding subscore of 0, an improvement in the endoscopic index of severity, a reduction in calprotectin in the stool to levels below 100 μg/g, improvement in quality of life as measured via the short irritable bowel disease questionnaire, a reduction in inflammatory immune cells in the colonic mucosa such as CD8+ T-cells, Th17 cells or neutrophils, a reduction in systemic or colonic inflammatory cytokines such as TNFa or IL-17, an increase in systemic or colonic anti-inflammatory cytokines such as IL-10 or IL-22, a reduction in systemic or colonic complement factors such as C5a or a normalization of the intestinal or conic microbiota.

In another embodiment, it is contemplated to administer a composition comprising indigo and/or an indigo derivative that does not include one or more of indole, indole-3-aldehyde, tryptanthrin, botulin, beta-sitosterol, daucosterol, indicant, and anthranilic acid to achieve and/or maintain a clinical response.

In still another embodiment, it is contemplated to provide a companion diagnostic together with a composition comprising indigo and/or an indigo derivative, wherein the diagnostic provides an indicator useful to titrate dose of the composition to achieve a non-therapeutic blood level.

In still another embodiment, it is contemplated to provide a composition comprising solely the E-stereoisomer of indirubin to achieve and/or maintain a clinical response.

In yet another embodiment, it is contemplated to provide a composition comprising solely the Z-stereoisomer of indirubin to achieve and/or maintain a clinical response.

In yet another embodiment, it is contemplated to provide a composition a mixture of the E-stereoisomer and Z-stereoisomer of indirubin.

In still another aspect, a method for preventing recurrence of UC is provided. The method comprises administering to a subject a therapeutically effective amount of a composition comprising indigo, indirubin, isatin, or a combination thereof, wherein the composition is not Indigo naturalis, whereby the administering prevents recurrence of UC in the subject.

Ulcerative colitis (UC) is a chronic, relapsing and remitting inflammatory bowel disease of unknown etiology that affects the colon and rectum in a circumferential and continuous manner. Depending on the extent of involvement, UC is classified as proctitis, proctosigmoiditis, left-sided colitis or pancolitis. Common symptoms include bloody diarrhea, fecal urgency and abdominal pain. UC is often treated with mesalamine, glucocorticoids, azathioprine, anti-tumor necrosis factor (TNF) agents (infliximab and adalimumab), 5-aminosalicylate drugs (e.g., Delzicol, Asacol, Pentasa, etc.), corticosteroids, Janus kinase (JAK) inhibitors, such as tofacitinib, peficitinib and filgotinib, other immunosuppressive agents and other biologic agents.

Inflammation in UC subjects is typically limited to the mucosa and extends from the rectum proximally. Crohn's disease, another inflammatory bowel disease, is characterized by transmural inflammation in any part of the gastrointestinal tract. In one embodiment, the compositions described herein are contemplated for treating an inflammatory bowel disease. In another embodiment, the compositions described herein are contemplated for treating an inflammatory bowel disease that is not Crohn's disease.

It will be appreciated that the compositions described herein are contemplated for treating other inflammatory conditions, such as hidradenitis suppurativa (HS). HS is a chronic inflammatory skin condition involving the follicular portion of folliculopilosebaceous units of apocrine gland-bearing skin. Patients can present with recurrent nodules, sinus tracts formation, abscesses, and/or scarring. The disease can manifest anywhere there are apocrine glands or hair follicles including the underarm, groin, buttocks and under the breasts. Symptoms may include pus-filled papules, cysts or nodules which may be painful and often emit an off-putting smell. For treating inflammatory skin conditions, the composition is a topical composition that is applied to the skin, in various embodiments, at least once daily, or at least two, three or four times daily. In one embodiment, the topical formulation is applied at least once daily for a period of about 4-12 weeks.

In other embodiments, the compositions are contemplated for use in treating a subject that has mild to moderate Crohn's disease or moderate to severe Crohn's disease. In other embodiments, the compositions are contemplated for use in treating a subject gastrointestinal graft-vs-host disease, pouchitis, or mucositis.

The dosing schedule for treating UC, IBD or other inflammatory disorders will depend, as can be appreciated, by factors well known in medical arts, including the dose of drug compound in the formulation, the severity of the disorder, and the health of the patient. In some embodiments, the methods comprise administering the composition at least about once daily, at least about twice daily, at least three times daily, or once daily, twice daily or three times daily. In one embodiment, the composition is administered for a period of at least about 1 week, 2 weeks, 4 weeks, 6 weeks, 8 weeks, 10 weeks, 12 weeks, 16 weeks, 20 weeks, 24 weeks, 52 weeks or chronically.

In other examples, formulations that result in systemic exposure may be used to treat other autoimmune or auto-inflammatory diseases. These include but are not limited to ankylosing spondylitis, atopic dermatitis, Alzheimer's disease, celiac disease, grave's disease, lupus, multiple sclerosis, psoriasis, rheumatoid arthritis, Sjögren's syndrome, type-1 and type-2 diabetes, vitiligo, or any of the diseases identified by the American Autoimmune Related Diseases Association in its Autoimmune Disease list viewable at aarda.org.

In other examples formulations may treat oral diseases or diseases with oral manifestations such as bacterial-related diseases such as syphilis, Behcet's disease, chronic aphthous stomatitis, erythema multiforme, eosinophillic esophagitis, MAGIC syndrome, mucous membrane pemphigoid, mucosal pemphigus vulgaris, Sweet syndrome or Wegner's Granulomatosis.

In other examples, the compositions (e.g., the dosage form and formulations) may be used to treat or prevent mucosal complications related to radiation therapy or chemotherapy such as chronic hemorrhagic radiation proctitis, diarrhea, xerostomia, mucositis, or dermatitis. In other examples, the compositions (e.g., the dosage form and formulations) may be used to treat or prevent complications related to organ transplant, such as gastrointestinal graft-vs-host disease.

In other examples, a higher dose is administered for initiation of treatment and a lower dose is administered for maintenance, in order to minimize long-term systemic exposure to the drug compounds. In one example, the initiation dose would be taken for about 8 weeks or until the patient's symptoms abate and/or go into remission, followed by administration of a maintenance dose that is less than the initiation dose. In some embodiments, a patient may increase the dose for a period of time corresponding to a flare in the disease, and then return to a lower maintenance dose.

In other examples the oral dosage form or jar containing the oral dosage form may include a sensor that works as part of a compliance management system alerting the patient, pharmacist or physician via smartphone application or text message when to take a dose or in the event of a missed dose or when medication is running low.

In other examples the dosage given to a patient or a patient's eligibility for use would be determined after genotyping or screening a patient for polymorphisms in the ligand binding domain of the aryl hydrocarbon receptor or other cellular components needs for effective aryl hydrocarbon signaling.

### EXAMPLES

The following examples are illustrative in nature and are in no way intended to be limiting.

### Example 1

### Exemplary Composition

50 mg of indigo is extracted from Indigo naturalis and placed into a gelatin capsule along with magnesium stearate and microcrystalline cellulose for oral delivery.

### Example 2

### Exemplary Composition

The compounds indigo and indirubin are synthetically prepared. The compounds are combined at a weight ratio of 60:40 and pressed into a solid core that is coated with an enteric polymer for oral delivery.

### Example 3

### Exemplary Composition

250 mg of tapinarof is synthesized and combined with 2 g of 5-ASA and pressed into a solid core along with an extended releases polymer of ethyl cellulose and the material is then coated with a pH sensitive coating made of polyvinyl acetate phthalate for oral administration.

### Example 4

### Exemplary Composition

500 mg of isatin is synthetically prepared in 1.5 grams of hydrogenated vegetable oil and pressed into an oblong cylinder for rectal administration.

### Example 5

### Exemplary Composition

1 mg of the E-isomer of indirubin in 1 g dextrose, microcrystalline cellulose, polyvinylpyrrolidone and magnesium stearate are mixed to produce a lozenge for oral administration.

### Example 6

### Exemplary Composition

2 mg of synthetic indirubin in the form of an amorphous solid suspension made of copovidone is mixed into a 500 mg lozenge made of dextrose, microcrystalline cellulose, polyvinylpyrrolidone and magnesium stearate for the treatment of Sorjen's syndrome or eosinophilic esophagitis.

### Example 7

### Exemplary Composition

0.25 mg of synthetic indirubin in the form of a partially amorphous solid suspension made of polyvinyl acetate and polyvinylcaprolactame-based graft copolymer (PVAc-PVCap-PEG, SOLUPLUS®) made via hot melt extrusion is mixed into swellable gasto-retentive tablet that slowly releases indirubin into the stomach for the treatment of autoimmune atrophic gastritis or gastic ulcers.

### Example 8

### Exemplary Composition

5 mg of synthetic indirubin in the form of a fully amorphous solid suspension made of hydroxypropylmethylcellulose phthalate (HPMCP) made via precipitation into an aqueous medium followed by dehydration in a vacuum oven is compressed into an enteric coated tablet for the treatment of Crohn's disease or Celiac's disease.

### Example 9

### Exemplary Composition

0.5 mg of synthetic indirubin in the form of an amorphous solid suspension made of hydroxypropylmethylcellulose phthalate (HPMCP HP55 is spray dried into 20 micron particles and compressed into a tablet with excipients, which is subsequently coated with methacrylic acid-ethyl acrylate copolymer (EUDRAGIT® L100-55) to form a colon-targeted delayed release tablet for the treatment of ulcerative colitis.

### Example 10

### Exemplary Composition

10 mg of synthetic indirubin is made in the form of an amorphous solid suspension made of a 1:1 mixture of povidone and HPMC HP55 is micronized and then suspended in an aersolizable foam and placed into a delivery device that can be inserted into the rectum to apply the foam for the treatment of proctitis or distal colitis.

### Comparative Example 1

### Amorphous Solid Dispersions of Indirubin in Polyvinylpyrrolidone

Synthetic crystalline indirubin was prepared as described in the literature (Wang, C. et al., Tetrahedron, 73: 2780-2785 (2017; Bergman, J. et al., J. Heterocyclic Chemistry, 1-10 (2014); Klock, C. et al., Bioorganic & Medicinal Chemistry Letters, 21: 2692-2696 (2011)) and was dissolved in tetrahydrofuran by warming to 50° C. to form a solution with indirubin at a concentration of 1 mg/mL. Separately, polyvinylpyrrolidone (PLASDONE™ K29/32 was dissolved in an 80/20 ethanol/water mixture to form a 4.5% PVP polymer solution.

Solid Dispersion 1: The indirubin solution and the polymer solution were mixed to provide a mixture with an indirubin:PVP weight ratio of 1:9. Solvent was removed by rotary evaporation at 50° C. The resulting film was clear in color, indicating none of the indirubin was in dissolved in the solid PVP, and crystalline drug was observed. An image of the film is shown in FIG. 1A.

Solid Dispersion 2: The indirubin solution and the polymer solution were mixed to provide a mixture with an indirubin:PVP ratio of 1:9. Solvent was reduced to 45% of original weight by rotary evaporation at 50° C. The mixture was placed in a petri dish and dried under vacuum at 50° C. The resulting film was clear in color, indicating none of the indirubin was in dissolved in the solid PVP, and crystalline drug was observed. An image of the film is shown in FIG. 1B.

### Example 11

### Amorphous Solid Dispersions of Indirubin in Polyvinylpyrrolidone

Synthetic crystalline indirubin was prepared as described in Comparative Example 1. It was dissolved in tetrahydrofuran by warming to 50° C. to form a solution with indirubin at a concentration of 1 mg/mL.

Solid Dispersion 3: Polyvinylpyrrolidone (“PVP”; PLASDONE™ K29/32) was dissolved in an 95% ethanol to form a 15% PVP polymer solution. The indirubin solution and the polymer solution were mixed to provide a mixture with an indirubin:PVP weight ratio of 1:9. Solvent was reduced to 45% of original weight by rotary evaporation at 50° C. The mixture was placed in a petri dish and dried under vacuum at 50° C. The resulting film was purplish in color, indicating some indirubin was in dissolved in the solid PVP and in the form of a solid solution.

Solid Dispersion 4: PVP (PLASDONE™ K29/32) was dissolved in anyhdrous methanol to form a 15% PVP polymer solution. The indirubin solution and the polymer solution were mixed to provide a mixture with an indirubin:PVP weight ratio of 1:9. Solvent was reduced to 45% of original weight by rotary evaporation at 50° C. The mixture was placed in a petri dish and dried under vacuum at 50° C. The resulting film was purplish in color, indicating some indirubin was in dissolved in the solid PVP and in the form of a solid solution. An image of the film is shown in FIG. 2A.

Solid Dispersion 5: PVP (PLASDONE™ K29/32 was dissolved in anyhdrous methanol to form a 15% PVP polymer solution. The indirubin solution and the polymer solution were mixed to provide a mixture with an indirubin:PVP ratio of 1:9. Solvent was removed by rotary evaporation at 50° C. The resulting film was purple with no crystalline indirubin observed indicating indirubin was in dissolved in the solid PVP and in the form of a solid solution. An image of the film is shown in FIG. 2B.

### Example 12

### Amorphous Solid Dispersions of Indirubin

Synthetic crystalline indirubin was prepared as described in Comparative Example 1. It was dissolved in tetrahydrofuran by warming to 50° C. to form a solution with indirubin at a concentration of 1 mg/mL.

The polymers listed below were separately dissolved in anyhdrous methanol to form a 15% polymer solution. The indirubin solution and the polymer solution were mixed to provide a mixture with an indirubin:polymer ratio of 1:9. Solvent was removed by rotary evaporation at 50° C. The polymers were butylated methacrylate copolymer (EUDRAGIT® EPO), methacrylic acid-ethyl acrylate copolymer (1:1) (EUDRAGIT® L100-55), PVP (KOLLIDON® VA 74), hydroxypropyl methylcellulose acetate succinate (HPMC-AS 912 F), hydroxypropylmethylcellulose phthalate (HPMCP-HP55), polyethylene oxide (POLYOX™ WSR-N10), polyvinyl acetate phthalate (PHTHALA VIN™), poloxamer P188 (KOLLIPHOR P188) and polyvinyl acetate and polyvinylcaprolactame-based graft copolymer (PVAc-PVCap-PEG, SOLUPLUS®).

The resulting films were observed for color and presence of crystalline indirubin. The films prepared with PVAc-PVCap-PEG and with polyvinylpyrrolidone-vinyl acetate copolymer (copovidone) were purple in color and with no crystalline indirubin observed, indicating indirubin was in dissolved in the solid polymer in amorphous form and in the form of a solid solution.

### Example 13

### Amorphous Solid Dispersions of Indirubin

A study was designed to evaluate influence of the solvent on amorphous dispersion formation. The polymers from Example 12 that possessed high degrees of crystallization were solubilized in various solvent systems (identified in Table 13-1). The polymer solutions were then added to solutions of indirubin in tetrahydrofuran to produce 9:1 polymer:indirubin ratios in the solvent systems (consisting of approximately 90% tetrahydrofuran and 10% polymer dissolution solvent). Films were cast from the solutions, dried by rotary evaporation, and then examined for the presence/absence of crystalline drug substance. The polymers, solvent systems, and observation from visual inspection for crystalline drug are set forth in Table 13-1.

TABLE 13-1

| Sample # | Polymer | Solvent | Description of Solid Dispersion |
|---|---|---|---|
| 1-1 | Polyox WSR | Acetontrile | Heavy recrystallization of indirubin drug |
| | N10 | | substance throughout dispersion |
| 1-2 | Polyox WSR | Methylene Chloride | Heavy recrystallization of indirubin drug |
| | N10 | | substance throughout dispersion. Smaller |
| | | | crystals present in the center of the |
| | | | dispersion and heavier recrystallization at |
| | | | the margins of the dispersion relative to the |
| | | | Polyox/ACN preparation. |
| 1-3 | HPMCP HP55 | Acetone:Methanol | Indirubin drug substance does not appear to |
| | | (1:1) | have recrystallized in the center of the |
| | | | dispersion. Rare instances of drug substance |
| | | | recrystallization are seen at margins of the |
| | | | dispersion. |
| 1-4 | HPMCP-HP55 | Ethyl | Indirubin drug substance does not appear to |
| | | Acetate:Methanol | have recrystallized in the center of the |
| | | (1:1) | dispersion. Recrystallization at the |
| | | | dispersion margins is more prevalent than in |
| | | | the HPMCP/Acetone Methanol sample. |
| 1-5 | HPMC-AS | Acetone | Light recrystallization of indirubin drug |
| | | | substance at center of dispersion with |
| | | | heavier recrystallization at the margins. |
| 1-6 | Eudragit | Acetone | Sparse recrystallization of indirubin drug |
| | L100-55 | | substance at center of dispersion with |
| | | | heavier recrystallization at the margins |
| 1-7 | Eudragit | Methanol | Indirubin drug substance does not appear to |
| | L100-55 | | have recrystallized in the center of the |
| | | | dispersion. Rare instances of drug substance |
| | | | recrystallization are seen at the margins of |
| | | | the dispersion. |
| 1-8 | Eudragit | Isopropanol | Heavy recrystallization of indirubin drug |
| | L100-55 | | substance throughout the dispersion. |
| 1-9 | Eudragit EPO | Acetone | Sparse recrystallization of indirubin drug |
| | | | substance at center of dispersion with |
| | | | heavier recrystallization at the margins. |
| 1-10 | Eudragit EPO | Methanol | Sparse recrystallization of indirubin drug |
| | | | substance at center of dispersion with |
| | | | heavier recrystallization at the margins. |
| 1-11 | Eudragit EPO | Isopropanol | Moderate recrystallization of indirubin drug |
| | | | substance at center of dispersion with |
| | | | heavier recrystallization at the margins. |
| 1-12 | Polyvinyl | Methanol | Sparse recrystallization of indirubin drug |
| | Acetate | | substance at center of dispersion with |
| | Phthalate | | heavier recrystallization at the margins. |

Samples 1-3, 1-5, 1-7, and 1-12 had low crystalline content. These polymer and solvents were used to in a further study using a 20/80 or a 50/50 polymer solvent/tetrahydrofuran ratio while maintaining the 9:1 polymer:indirubin ratio. Films cast from these materials were examined for the prevalence of crystalline materials and the results of these evaluations is in Table 13-2.

TABLE 13-2

| Sample # | Polymer | Solvent | Description of Solid Dispersion |
|---|---|---|---|
| 1-3 | HPMCP HP55 | 20% | Indirubin drug substance does not appear to |
| | | Acetone:Methanol | have recrystallized in the center of the |
| | | (1:1) 80% THF | dispersion. Drug substance crystals at the |
| | | | margins of the dispersion are smaller and |
| | | | more rare than seen in the 10% polymer |
| | | | solvent preparation. |
| 1-3 | HPMCP HP55 | 20% | Indirubin drug substance does not appear to |
| | | Acetone:Methanol | have recrystallized in the center of the |
| | | (1:1) 50% THF | dispersion. Drug substance crystals at the |
| | | | margins of the dispersion are small and very |
| | | | rare compared to 10% polymer solvent |
| | | | preparation with slightly less recrystallization |
| | | | than the 20% polymer solvent preparation. |
| 1-5 | HPMC-AS | 20% Acetone | Indirubin drug substance does not appear to |
| | | 80% THF | have recrystallized in the center of the |
| | | | dispersion. Drug substance crystals at the |
| | | | margins of the dispersion are smaller and |
| | | | more rare than seen in the 10% polymer |
| | | | solvent preparation. |
| 1-5 | HPMC-AS | 50% Acetone | Indirubin drug substance does not appear to |
| | | 50% THF | have recrystallized in the center of the |
| | | | dispersion. Drug substance crystals at the |
| | | | margins of the dispersion are very small and |
| | | | vary rare compared to 10% and 20% polymer |
| | | | solvent preparations. |
| 1-7 | Eudragit | 20% Methanol | Indirubin drg substance does not appear to |
| | L100-55 | 80% THF | have recrystallized in the center of the |
| | | | dispersion. Drug substance crystals at the |
| | | | margins of the dispersion are considerably |
| | | | smaller than seen in the 10% polymer solvent |
| | | | preparation |
| 1-7 | Eudragit | 50% Methanol | Indirubin drug substance does not appear to |
| | L100-55 | 50% THF | have recrystallized in the center of the |
| | | | dispersion. Drug substance crystals at the |
| | | | margins of the dispersion are larger and more |
| | | | numerous than seen in the 20% polymer |
| | | | solvent preparation |
| 1-12 | Polyvinyl | 20% Methanol | Indirubin drug substance does not appear to |
| | Acetate | 80% THF | have recrystallized in the center of the |
| | Phthalate | | dispersion. Drug substance crystals at the |
| | | | margins of the dispersion are smaller and less |
| | | | numerous than seen in the 10% polymer |
| | | | solvent preparation |
| 1-12 | Polyvinyl | 50% Methanol | Indirubin drug substance does not appear to |
| | Acetate | 50% THF | have recrystallized in the center of the |
| | Phthalate | | dispersion. Drug substance crystals at the |
| | | | margins of the dispersion are larger and more |
| | | | numerous than seen in the 20% polymer |
| | | | solvent preparation, but smaller and less |
| | | | numerous than seen in the 10% polymer |
| | | | solvent preparation. |

### Example 14

### X-Ray Diffraction of Solid Amorphous Dispersions of Indirubin

Solid dispersions with synthetic indirubin were prepared for analysis by x-ray diffraction (XRD). Films of solid dispersions were cast directly onto zero background silicon sample holder slides. Films of indirubin:povidone dispersions were prepared at 10% and 5% indirubin load using the tetrahydrofuran/methanol solvation/evaporation preparation technique described in Example 11, Solid Dispersion 5. All other indirubin dispersions for this study were prepared at either 5% or 10% drug load as described in Example 13. All films were analyzed by x-ray diffraction, and the diffractograms are shown in FIGS. 3C-3M. FIG. 3A is an x-ray diffractogram for the zero background holder and FIG. 3B is an x-ray diffractogram for neat synthetic indirubin in tetrahydrofuran at 1 mg/mL, added to the zero background holder dropwise and allowed to dry completely and represent the expected results of a 10% indirubin load dispersion with 100% crystalline drug substance. The tables below summarize the properties of the diffractograms.

TABLE 14-1
Analysis of XRD Diffractogram for Empty Zero Background Sample Holder (FIG. 3A)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 3.31(3) | 26.7(2) | 643(15) | 7.34(12) | 7313(271) | 11.4(7) | 5(12) |
| 2 | 9.51(2) | 9.29(2) | 98(6) | 1.32(3) | 139(5) | 1.42(13) | 1.53(11) |
| 3 | 14.56(7) | 6.08(3) | 48(4) | 4.2(4) | 412(93) | 9(3) | 2.0(10) |
| 4 | 23.7(2) | 3.75(3) | 16(2) | 4.5(7) | 123(34) | 8(3) | 2.9(10) |
| 5 | 36.98(14) | 2.429(9) | 42(4) | 5.5(4) | 246(35) | 5.8(13) | 0.6(3) |

TABLE 14-2
Analysis of XRD Diffractogram for 100% Crystalline Indirubin
(at Concentration Equivalent to 10% Drug Load Samples) (FIG. 3B)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 8.422(3) | 10.490(4) | 1075(19) | 0.147(2) | 182(5) | 0.169(7) | 1.17(9) |
| 2 | 9.29(3) | 9.52(3) | 106(6) | 1.46(7) | 177(5) | 1.68(15) | 1.17(9) |
| 3 | 11.00(18) | 8.04(13) | 66(5) | 0.28(4) | 21(3) | 0.32(7) | 1.17(9) |
| 4 | 11.150(4) | 7.929(3) | 294(10) | 0.159(8) | 54(3) | 0.183(15) | 1.17(9) |
| 5 | 14.626(7) | 6.051(3) | 363(11) | 0.214(7) | 118.9(19) | 0.327(15) | 1.3(2) |
| 6 | 16.967(4) | 5.2215(13) | 318(10) | 0.174(3) | 65.5(11) | 0.206(10) | 1.02(10) |
| 7 | 18.50(4) | 4.792(9) | 30(3) | 0.21(3) | 6.6(12) | 0.22(6) | 1.2(8) |
| 8 | 23.78(3) | 3.739(4) | 28(3) | 0.80(7) | 46.3(19) | 1.7(3) | 2.8(6) |
| 9 | 25.684(13) | 3.4657(17) | 16(2) | 0.25(4) | 4.9(7) | 0.30(9) | 3(4) |
| 10 | 34.420(10) | 2.6035(7) | 35(3) | 0.34(6) | 23.4(11) | 0.68(10) | 0.5(3) |
| 11 | 36.78(3) | 2.4415(19) | 27(3) | 3.09(7) | 89(2) | 3.3(5) | 0.98(15) |

TABLE 14-3
Analysis of XRD Diffractogram of 10% indirubin in PVP dispersion (FIG. 3C)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 8.429(4) | 10.481(5) | 441(12) | 0.142(4) | 67(2) | 0.152(10) | 1.54(15) |
| 2 | 9.51(3) | 9.30(3) | 92(6) | 1.33(6) | 131(4) | 1.42(13) | 1.54(15) |
| 3 | 10.98(5) | 8.05(3) | 58(4) | 0.21(5) | 13(5) | 0.23(11) | 1.54(15) |
| 4 | 11.186(4) | 7.903(3) | 448(12) | 0.161(11) | 77(6) | 0.172(17) | 1.54(15) |
| 5 | 14.624(4) | 6.0524(17) | 413(12) | 0.202(4) | 117.2(17) | 0.284(12) | 1.00(9) |
| 6 | 15.190(19) | 5.828(7) | 21(3) | 0.26(5) | 7.8(13) | 0.37(11) | 1.00(9) |
| 7 | 16.958(8) | 5.224(2) | 132(7) | 0.158(9) | 29.9(8) | 0.227(18) | 0.69(18) |
| 8 | 18.609(5) | 4.7644(11) | 46(4) | 0.199(9) | 10.4(4) | 0.23(3) | 4.5(7) |
| 9 | 23.678(9) | 3.7546(13) | 33(3) | 0.27(2) | 9.9(11) | 0.30(6) | 0.5(3) |

TABLE 14-4
Analysis of XRD Diffractogram of 5% indirubin in PVP dispersion (FIG. 3D)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 9.59(4) | 9.22(4) | 197(8) | 2.51(8) | 952(39) | 4.8(4) | 3.0(3) |
| 2 | 11.12(6) | 7.95(4) | 108(6) | 2.12(19) | 446(62) | 4.1(8) | 1.1(3) |
| 3 | 14.04(13) | 6.30(6) | 65(5) | 4.6(5) | 474(93) | 7.3(19) | 1.1(11) |
| 4 | 22.20(10) | 4.001(18) | 79(5) | 10.12(16) | 1144(73) | 14.5(19) | 0.50(3) |
| 5 | 37.49(4) | 2.397(3) | 41(4) | 6.2(3) | 506(45) | 12(2) | 3.2(6) |

TABLE 14-5
Analysis of XRD Diffractogram of 10% indirubin in polyvinylpyrrolidone-vinyl
acetate copolymer (copovidone) dispersion from THF/methanol (FIG 3E)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 9.59(5) | 9.22(5) | 137(7) | 3.74(14) | 1013(57) | 7.4(8) | 5.0(7) |
| 2 | 11.10(4) | 7.96(3) | 62(5) | 0.41(9) | 54(13) | 0.9(3) | 0.5(3) |
| 3 | 12.6(8) | 7.0(4) | 60(4) | 18.1(10) | 2238(76) | 37(4) | 0.5(5) |
| 4 | 14.67(5) | 6.035(18) | 44(4) | 1.01(18) | 95(19) | 2.1(6) | 3.7(11) |
| 5 | 21.86(11) | 4.06(2) | 27(3) | 5.5(2) | 175(10) | 6.4(11) | 0.51(12) |

TABLE 14-6
Analysis of XRD Diffractogram of 5% indirubin in in polyvinylpyrrolidone-vinyl
acetate copolymer (copovidone) dispersion from THF/Methanol (FIG. 3F)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 9.3(2) | 9.48(19) | 117(6) | 6.8(5) | 1698(189) | 14(2) | 1.3(3) |
| 2 | 20.75(16) | 4.28(3) | 120(6) | 8.5(4) | 1675(157) | 14(2) | 0.74(10) |

TABLE 14-7
Analysis of x-ray diffraction diffractogram of a dispersion of 10% indirubin in
HPMC-AS912 Dispersion from THF and Methanol/Acetone (FIG. 3G)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 8.4(2) | 10.5(3) | 146(7) | 6.3(3) | 1710(575) | 12(4) | 0.50(11) |
| 2 | 24(20) | 3.7(17) | 51(4) | 276(1013) | 21207(396) | 413(41) | 1(3534) |

TABLE 14-8
Analysis of x-ray diffraction diffractogram of a dispersion of 5% indirubin in
HPMC-AS912 dispersion from THF and Methanol/Acetone (FIG. 3H)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | −2.12(9) | 0 | 71932(155) | 9.09(18) | 901981(960024) | 13(13) | 3(91940) |
| 2 | 9.28(7) | 9.52(7) | 345(11) | 3.4(4) | 1812(524) | 5.3(17) | 0.61(15) |
| 3 | 18.97(19) | 4.67(4) | 264(9) | 10.6(8) | 4135(481) | 16(2) | 1.37(16) |
| 4 | 36.86(12) | 2.436(7) | 131(7) | 12.3(8) | 2420(196) | 18(2) | 4(7) |

TABLE 14-9
Analysis of x-ray diffraction of a dispersion of 10% indirubin in
HPMCP (HP55) from THF and Acetone/Methanol (FIG. 3I)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 2.09(14) | 42(3) | 759(16) | 6.2(3) | 8228(808) | 10.8(13) | 2(113374) |
| 2 | 8.8(11) | 10.0(11) | 43(4) | 13(2) | 1170(381) | 27(11) | 3(992) |
| 3 | 9.15(5) | 9.65(5) | 199(8) | 10(5) | 3963(2011) | 20(11) | 4(4062) |
| 4 | 20(3) | 4.5(5) | 153(7) | 15(12) | 2363(61) | 15.5(11) | 1.17(10) |

TABLE 14-10
Analysis of x-ray diffraction of a dispersion of 5% indirubin in HPMCP
(HP55) from THF and Acetone/Methanol (FIG. 3J)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | — | 0(32) | 0.0000( | 185(117629331 | 0(133) | 254(19170079 | 1(1772500 |
| 2 | 1.57(3) | 56.3(1 | 1820(25 | 2.50(7) | 5213(139 | 2.9(8) | 1(182624) |
| 3 | 8.80(4) | 10.04( | 279(10) | 3.49(4) | 1085(10) | 3.88(17) | 1.32(5) |
| 4 | 19.32(2) | 4.589( | 212(8) | 8.67(6) | 2221(16) | 10.5(5) | 0.84(6) |

TABLE 14-11
Analysis of x-ray diffraction of a dispersion of 10% indirubin in polyvinyl
acetate phthalate (PVAP) from THF/methanol (FIG. 3K)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 8.372(4) | 10.553(5) | 174(8) | 0.140(11) | 34(3) | 0.20(2) | 0.50(4) |
| 2 | 9.260(15) | 9.542(15) | 101(6) | 0.99(4) | 141(4) | 1.39(12) | 0.50(4) |
| 3 | 11.146(2) | 7.9316(16) | 259(9) | 0.113(6) | 41.1(13) | 0.159(11) | 0.50(4) |
| 4 | 14.585(8) | 6.068(3) | 264(9) | 0.155(8) | 64.1(13) | 0.243(14) | 1.5(4) |
| 5 | 16.926(8) | 5.234(3) | 37(4) | 0.166(14) | 7.1(5) | 0.19(3) | 0.85(18) |
| 6 | 18.598(10) | 4.767(3) | 21(3) | 0.25(3) | 6.2(9) | 0.29(8) | 3(3) |

TABLE 14-12
Analysis of x-ray diffraction of a dispersion of 5% indirubin in polyvinyl
acetate phthalate (PVAP) from THF/methanol (FIG. 3L)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 10(2477) | 9(7) | 51(4) | 713(26885569) | 71128(267692357) | 1394(5247074) | 5(159999) |
| 2 | 15(872) | 6(5) | 83(5) | 713(192264) | 115040(890131) | 1394(10876) | 5(402782) |

TABLE 14-13
Analysis of x-ray diffraction of a dispersion of 10% indirubin in PVAc-PVCap-PEG,
(SOLUPLUS ^{®}) from THF (FIG. 3M)

| No. | 2-theta(deg) | d(ang.) | Height(cps) | FWHM(deg) | Int. I(cps deg) | Int. W(deg) | Asym. factor |
|---|---|---|---|---|---|---|---|
| 1 | 7.8(15) | 11.4(19) | 34212(107) | 57(310) | 2187192(343148) | 64(10) | 1(5) |
| 2 | 8.40(9) | 10.52(11) | 820(17) | 6.5(3) | 5964(970) | 7.3(13) | 0.73(8) |

### Example 15

### Release of Indirubin from Solid Amorphous Dispersions

Solid amorphous dispersions of synthetic indirubin in six different hydrophilic carrier polymers-povidone, PVAc-PVCap-PEG, (SOLUPLUS®), copovidone, HPMC-AS, HPMCP and polyvinyl acetate phthalate (PVAP) with indirubin to polymer ratios of 1:9 or 0.5 to 9.5 were prepared as described in Example 13. For comparison, Indigo naturalis and crystalline synthetic indirubin were also tested. All test articles were normalized to a total indirubin of 2 mg and added to 500 mL of fasted-state simulated intestinal fluid (FaSSIF) medium consisting of 3 mM taurocholate, 0.75 mM phospholipids, 148 mM sodium, 106 mM chloride, and 29 mM phosphate at pH 6.5. Six replicates of each test article were assessed under the following testing conditions: mixing with USP #2 paddles at 100 RPM at 37° C. with sampling of 5 mL at each time point and filtration through a 10 μm filter. The amount of indirubin in solution was determined with an HPLC method specific for indirubin that did not display interference from indigo or the various polymers. The mean amount of indirubin in solution at each time point was determined, and is shown in Table 1 in units of μg/L and in Table 2 as a percentage, referred to a % Q, of measured amount (mass) of indirubin in solution divided by the theoretical maximum amount (mass) of indirubin in solution.

### Example 16

### Comparison of Carbon Content from Synthetic Vs. Natural Indirubin

A synthetic sample of indirubin made from a reaction of isatin with PCLs followed by a subsequent reaction with oxindole in toluene resulted in indirubin following work up that was greater than 97% pure via HPLC. Naturally occurring indirubin extracted from Baphicacanthus cusia that was also greater than 97% pure utilizing the same HPLC method. Both the synthetic and natural indirubin were analyzed for their percent ¹⁴C carbon content in parts per trillion (ppt). Data was normalized and presented as units of percent fossil carbon content in line with the ASTM D6866 procedure. The percent of ¹⁴C provides a measure of the amount of carbon in the molecule originating from fossilized hydrocarbon based starting materials which are expected to have a very low ¹⁴C content in comparison to material isolated from recently living sources which would be expected to have close to an atmospheric amount of ¹⁴C. The amount of ¹⁴C in a sample changes slowly as the half-life of ¹⁴C is 5,730 years. The data is in Table 16-1.

TABLE 16-1

| | Fossil Carbon Content | | ¹⁴C Content | |
|---|---|---|---|---|
| Sample | % | error | ppt ¹⁴C | error |
| Synthetic indirubin | 87% | 3% | 0.13 | 0.03 |
| Natural indirubin | 0% | 3% | 1.00 | 0.03 |

Accordingly, in one embodiment this data provides a clear method for distinguishing between synthetic indirubin derived from fossilized hydrocarbon starting materials and naturally derived indirubin based on the ¹⁴C content. In one aspect synthetic indirubin has a ¹⁴C content of less than about 0.9 ppt ¹⁴C. In another aspect synthetic indirubin has a fossil carbon content of greater than 10%.

### Example 17

### In vitro AhR activation assay

Indirubin, indigo and Indio naturalis were incubated for 24 hours with reporter cells made from human Huh7 cells expressing human AhR and ARNT with luciferase expressed behind a genetic response elements that responds to dimerized AhR/ARNT. After incubation, the medium was aspirated and a detection reagent was added followed by a measurement of the relative luminescent units compared to a negative control of DMSO. Data is shown in FIG. 4.

### Example 18

### Treatment of UC with Oral Compositions

Patients with mild, moderate or severe UC are recruited for a study. The recruited patients are randomized into two groups for treatment with a solid oral dosage form made from a solid amorphous dispersion of synthetic indirubin in a hydrophilic polymeric carrier (Cohort 1) or with the plant extract Indigo naturalis (Cohort 2). Both cohorts self-administer the respective treatment orally twice daily for 12 weeks for a daily dose of 500 mg (Cohort 1:500 mg synthetic indirubin; Cohort 2:500 mg Indigo naturalis). The subjects are evaluated for disease activity using the partial and total Mayo scores, flexible sigmoidoscopy, calprotectin, and Short Inflammatory Bowel Disease Questionnaire (SIBDQ) at baseline and at weeks 2, 4, 8 and 12. The Mayo scoring system is set forth below.

All patients in Cohort 1 achieve clinical remission of UC after 12 weeks defined as a change in Mayo scored of at least 3 and a change of at least about 30% from baseline visit.

### Example 19

### Treatment of UC with Oral Compositions

Patients with mild, moderate or severe UC are recruited for a study. The recruited patients are randomized into two groups for treatment with a composition of synthetic indigo and indirubin (Cohort 1) or with 500 mg of the plant extract Indigo naturalis (Cohort 2). Both cohorts self-administer the respective treatment orally twice daily for 12 weeks for a daily dose of 500 mg (Cohort 1:300 mg indigo and 200 mg indirubin; Cohort 2:500 mg Indigo naturalis). The subjects are evaluated for disease activity using the partial and total Mayo scores, flexible sigmoidoscopy, calprotectin, and Short Inflammatory Bowel Disease Questionnaire (SIBDQ) at baseline and at weeks 2, 4, 8 and 12. TheMayo scoring system is set forth below.

All patients in Cohort 1 achieve clinical remission of UC after 12 weeks defined as a change in Mayo scored of at least 3 and a change of at least about 30% from baseline visit.

Mayo Scoring System for Assessment
of Ulcerative Colitis Activity*

Stool frequency ^{†}
0 = Normal no. of stools for this patient
1 = 1 to 2 stools more than normal
2 = 3 to 4 stools more than normal
3 = 5 or more stools more than normal
Subscore, 0 to 3
Rectal bleeding ^{‡}
0 = No blood seen
1 = Streaks of blood with stool less than half the time
2 = Obvious blood with stool most of the time
3 = Blood alone passes
Subscore, 0 to 3
Findings on endoscopy
0 = Normal or inactive disease
1 = Mild disease (erythema, decreased vascular pattern, mild friability)
2 = Moderate disease (marked erythema, lack of vascular pattern,
friability)
3 = Severe disease (spontaneous bleeding, ulceration)
Subscore, 0 to 3
Physician's global assessment ^{§}
0 = Normal
1 = Mild disease
2 = Moderate disease
3 = Severe disease
Subscore, 0 to 3
*The Mayo score ranges from 0 to 12, with higher scored indicating more severe disease.
^{†} Each patient serves as his or her own control to establish the degree of abnormality of the stool frequency.
^{‡} The daily bleeding score represents the most severe bleeding of the day.
^{§} The physician's global assessment acknowledges the 3 other observations, such as physical findings and patient's performance status.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. An orally-administrable dosage form, comprising:
an amount of an aryl hydrocarbon receptor (AhR) agonist and a polymer that is a copolymer selected from the group consisting of (i) a copolymer of methacrylic acid and methyl methacrylate, (ii) a copolymer of methacrylic acid and ethyl acrylate and (iii) a copolymer of methyl methacrylate, butyl methacrylate, and dimethylaminoethyl methacrylate, wherein the dosage form after oral administration delivers the AhR agonist to the small intestine or large intestine, and wherein the AhR agonist is an at least 20-fold more potent activator of AhR than Indigo naturalis on a gram for gram basis.

2. The dosage form of claim 1, wherein the aryl hydrocarbon receptor agonist is selected from the group consisting of a prodrug of an indigo derivative that decomposes or is metabolized into an indigo derivative and a prodrug of an indirubin derivative that decomposes or is metabolized into an indirubin derivative.

3. The dosage form of claim 1, comprising between about 0.5-20 wt % of the aryl hydrocarbon receptor agonist.

4. The dosage form of claim 1, wherein the dosage form is selected from the group consisting of an enteric-coated tablet and an enteric-coated tablet capsule.

5. The dosage form on claim 1, wherein the copolymer is a copolymer consisting of methacrylic acid and methyl methacrylate.

6. The dosage form of claim 5, wherein the copolymer has a 1:2 or a 1:1 ratio of methacrylic acid and methyl methacrylate.

7. The dosage form on claim 1, wherein the copolymer is a copolymer consisting of methacrylic acid and ethyl acrylate.

8. The dosage form of claim 1, wherein the aryl hydrocarbon receptor agonist is selected from the group consisting of tapinarof, indigo, an indigo derivative, a prodrug of indigo, and a prodrug of an indigo derivative.

9. An orally-administrable dosage form, comprising:
an amount of a prodrug of an indigo derivative that decomposes or is metabolized into an indigo derivative and a polymer that is a copolymer selected from the group consisting of (i) a copolymer of methacrylic acid and methyl methacrylate, (ii) a copolymer of methacrylic acid and ethyl acrylate and (iii) a copolymer of methyl methacrylate, butyl methacrylate, and dimethylaminoethyl methacrylate, wherein the dosage form after oral administration delivers the indigo derivative to the small intestine or large intestine, and wherein the indigo derivative is an at least 20-fold more potent activator of AhR than Indigo naturalis on a gram for gram basis.

10. A method for treating a gastro-intestinal inflammatory disease, comprising:
administering to a subject a dosage form comprising an amount of an aryl hydrocarbon receptor (AhR) agonist and a polymer that is a copolymer selected from the group consisting of (i) a copolymer of methacrylic acid and methyl methacrylate, (ii) a copolymer of methacrylic acid and ethyl acrylate and (iii) a copolymer of methyl methacrylate, butyl methacrylate, and dimethylaminoethyl methacrylate, wherein the dosage form after oral administration delivers the AhR agonist to the small intestine or large intestine, and wherein the AhR agonist is an at least 20-fold more potent activator of AhR than Indigo naturalis on a gram for gram basis.

11. The method of claim 10, wherein the gastro-intestinal inflammatory disease is Crohn's disease.

12. The method of claim 10, wherein the AhR agonist is at least 20-fold more potent activator of AhR than Indigo naturalis on a gram for gram basis in vivo.

13. A method for treating ulcerative colitis (UC) in a subject refractory or intolerant to a known therapy selected from the group consisting of 5-aminosalicylic acid, a prodrug of 5-aminosalicylic acid, a steroid, an anti-TNF-alpha antibody, peficitinib, tofacitinib, vedolizumab, and ozanimod, comprising:
administering to the subject a dosage form comprising an amount of an aryl hydrocarbon receptor (AhR) agonist and a polymer that is a copolymer selected from the group consisting of (i) a copolymer of methacrylic acid and methyl methacrylate, (ii) a copolymer of methacrylic acid and ethyl acrylate and (iii) a copolymer of methyl methacrylate, butyl methacrylate, and dimethylaminoethyl methacrylate, wherein the dosage form after oral administration delivers the AhR agonist to the small intestine or large intestine, and wherein the AhR agonist is an at least 20-fold more potent activator of AhR than Indigo naturalis on a gram for gram basis.

14. The method of claim 13, wherein the subject has moderate to severe UC or mild to moderate UC.

15. An orally-administrable dosage form, comprising:
an amount of an aryl hydrocarbon receptor (AhR) agonist suspended or dissolved without use of a solvent in a polymer to form a solid suspension used to prepare a dosage form, the dosage form coated with a pH-sensitive polymer, wherein the dosage form after oral administration delivers the AhR agonist to the small intestine or large intestine, and wherein the AhR agonist is an at least 20-fold more potent activator of AhR than Indigo naturalis on a gram for gram basis.

16. The dosage form of claim 15, wherein the pH-sensitive polymer is a copolymer of methacrylic acid and methyl methacrylate.

17. The dosage form of claim 15, wherein the pH-sensitive polymer is selected from the group consisting of poly(methacrylic acid-co-methyl methacrylate) 1:1; poly(methacrylic acid-co-ethyl acrylate) 1:1, poly(methacrylic acid-co-methyl methacrylate) 1:2, and a mixture of poly(methacrylic acid-co-methyl methacrylate) 1:1 and poly(methacrylic acid-co-methyl methacrylate) 1:2 in a ratio of 1:10 to 1:2.

18. The dosage form of claim 15, wherein the pH-sensitive polymer is a cellulosic polymer or copolymer.

19. The dosage form of claim 15, wherein the cellulosic polymer or copolymer is selected from the group consisting of hydroxypropyl methylcellulose acetate succinate, hydroxypropyl methylcellulose phthalate, hydroxypropyl methylcellulose and carboxymethyl ethylcellulose.

20. The dosage form of claim 15, further comprising an agent selected from the group consisting of hydroxypropylcellulose, hydroxypropylmethylcellulose, hydroxyethylcellulose, methylcellulose, organo/cold water soluble cellulose, hydroxyethylmethylcellulose, ethylcellulose, and ethyl(hydroxyethyl)cellulose.
